(19) United States
(12) Patent Application Publication
(10) Pub. No.: US 2011/0118274 A1
(43) Pub. Date: May 19, 2011

(54) PROTEASOME INHIBITORS AND THEIR USE IN TREATING PATHOGEN INFECTION AND CANCER

(75) Inventors: Carl Nathan, Larchmont, NY (US); Gang Lin, Forest Hills, NY (US)

(73) Assignee: CORNELL RESEARCH FOUNDATION, INC., Ithaca, NY (US)

(21) Appl. No.: 12/674,820

(22) PCT Filed: Aug. 25, 2008
(86) PCT No.: PCT/US08/74146

§ 371 Date: May 14, 2010

Related U.S. Application Data
(60) Provisional application No. 60/957,610, filed on Aug. 23, 2007.

(51) Int. Cl. A61K 31/41; A61K 31/422; A61K 31/427; A61K 31/4245; A61K 31/433; A61K 31/4439; A61K 31/501; A61K 31/506; A61K 31/497; C07D 419/04; A61P 35/00; A61P 31/06; A61P 31/00; A61P 31/04; A61P 31/08

(52) U.S. Cl. 514/252.05; 514/360; 514/378; 514/374; 514/365; 514/372; 514/364; 514/361; 514/362; 514/342; 514/256; 514/255.05; 548/122; 514/380; 514/376; 514/377; 514/369; 514/370

(57) ABSTRACT

The present invention relates to proteasome inhibitors and their use in methods of treating a subject for a pathogen infection or cancer. The methods involve administering to the subject a compound of Formula (I). (I) where: Q is Formula or Formula, where the crossing dashed line illustrates the bond formed joining Q to the rest of the compound of Formula (I). The remainder of substituents of the compound of Formula (I) are defined in the present application.

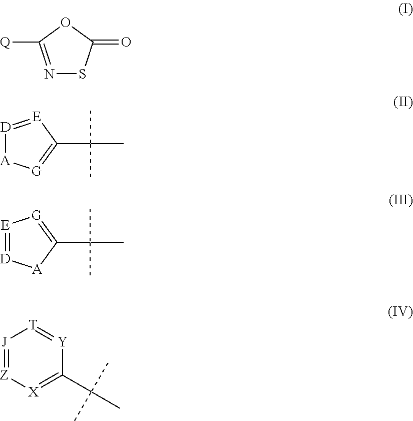

This application claims the benefit of U.S. Provisional Patent Application Ser. No. 60/957,610, filed Aug. 23, 2007.

The subject matter of this application was made with support from the United States Government under The National Institutes of Health, Grant No. POI A1056293. The government has certain rights in this invention.

### FIELD OF THE INVENTION

The present invention relates to proteasome inhibitors and their use in treating pathogen infection and cancer.

### BACKGROUND OF THE INVENTION

Mycobacterium tuberculosis (Mtb) is causing a global health emergency that is rapidly worsening through the intersection of the tuberculosis pandemic with epidemics of antibiotic resistance, HIV/AIDS, and obesity-associated diabetes (Corbett et al., “The Growing Burden of Tuberculosis: Global Trends and Interactions with the HIV Epidemic,”Arch. Intern. Med. 163:1009-1021 (2003); and Restrepo, B. I., “Convergence of the Tuberculosis and Diabetes Epidemics: Renewal of Old Acquaintances,” Clin. Infect. Dis. 45:436-438 (2007)). Yet little new chemotherapy against Mtb has emerged in decades. In 1931, Dubos and Avery (Avery et al., “The Protective Action of a Specific Enzyme Against Type III Pneumococcus Infection in Mice,” J. Exp. Med. 54:73-89 (1931)) introduced the concept of targeting a pathway in the pathogen. The pathway is essential for the pathogen survival in the host, even though the pathogen does not require that pathway for survival in a bacteriologic culture medium that supports its rapid replication. Perhaps because the antimicrobial agent used by Dubos and Avery was a microbial enzyme whose efficacy was abolished when the host developed antibodies against it, this approach was ignored for over 70 years, until the contemporary crisis in anti-infectives discovery led to its reconsideration (Nathan, C., “Antibiotics at the Crossroads,” Nature 431:899-902 (2004); and Clatworthy et al., “Targeting Virulence: A New Paradigm for Antimicrobial Therapy,” Nat. Chem. Biol. 3:541-548 (2007)) and demonstration with small molecules (Liu et al., “Staphylococcus aureus Golden Pigment Impairs Neutrophil Killing and Promotes Virulence Through its Antioxidant Activity,” J. Exp. Med. 202:209-215 (2005)).

The proteasome is not essential for Mtb survival under in vitro growth-sustaining conditions, but has emerged as an element in a pathway that Mtb requires in order to survive nitrosative stress and other conditions that the pathogen faces during the course of infection (Darwin et al., “The Proteasome of Mycobacterium tuberculosis is Required for Resistance to Nitric Oxide,” Science 302:1963-1966 (2003); and Gandotra et al., “In vivo Gene Silencing Identifies the Mycobacterium tuberculosis Proteasome as Essential for the Bacteria to Persist in Mice,” Nat. Med. 13:1515-1520 (2007)). Proteasomes are ubiquitous in eukaryotic cells, widespread in archaea and rare in eubacteria. In eukaryotes the proteasome core is a stack of 7 types of α subunits forming two heteroheptameric outer rings and 7 types of β subunits forming two heteroheptameric inner rings (Kisselev et al., “Proteasome Inhibitors: from Research Tools to Drug Candidates,” Chem. Biol. 8:739-758 (2001)). Three of the β subunits (β1—caspase-like, β2—trypsin-like, and β5—chymotrypsin-like) display proteolytic activity (Orlowski et al., “Evidence for the Presence of Five Distinct Proteolytic Components in the Pituitary Multicatalytic Proteinase Complex. Properties of Two Components Cleaving Bonds on the Carboxyl Side of Branched Chain and Small Neutral Amino Acids,” Biochemistry 32:1563-1572 (1993)). Prokaryotic proteasomes usually have only one type of α subunits and 1 or 2 types of β subunits. The γ-OH of the N-terminal threonine of the subunits forms a key element of each active site (Seemuller et al., “Autocatalytic Processing of the 20S Proteasome,” Nature 382:468-471 (1996)). Because there are few N-terminal threonine-dependent hydrolases, it has been possible to develop proteasome inhibitors that spare other proteases to various degrees (Kisselev et al., “Proteasome Inhibitors: from Research Tools to Drug Candidates,” Chem. Biol. 8:739-758 (2001); Goldberg et al., “Not Just Research Tools—Proteasome Inhibitors Offer Therapeutic Promise,” Nat. Med. 8:338-340 (2002)), such as epoxyketones (Kim et al., “Proteasome Inhibition by the Natural Products Epoxomicin and Dihydroeponemycin: Insights Into Specificity and Potency,” Bioorg. Med. Chem. Lett. 9:3335-3340 (1999)) and peptidyl boronic acids (Adams et al., “Potent and Selective Inhibitors of the Proteasome: Dipeptidyl Boronic Acids,” Bioorg. Med. Chem. Lett. 8:333-338 (1998)). Studies of the cytotoxicity of proteasome inhibitors have indicated a strong correlation between inhibitory activity against chymotryptic-like activity and their cytotoxicity (Adams et al., “Proteasome Inhibitors: A Novel Class of Potent and Effective Antitumor Agents,” Cancer Res. 59:2615-2622 (1999)). The peptidyl boronate bortezomib (Velcade®) is in clinical use for the treatment of multiple myeloma and other malignancies (Kropff et al., “Bortezomib in Combination With Intermediate-Dose Dexamethasone and Continuous Low-Dose Oral Cyclophosphamide for Relapsed Multiple Myeloma,” Br. J. Haematol. 138:330-337 (2007)).

Although Mtb is a bacterium, it has been shown that it expresses a proteasome core consisting of the typical four heptameric rings stacked in a cylinder. Cryoelectron microscopy, X-ray crystallography with a peptidyl boronate inhibitor and mutation analysis suggested that the α subunits have a gating function and confirmed that the β subunits provide the active site N-terminal threonine hydroxyl (Lin et al., “Mycobacterium tuberculosis prcBA Genes Encode a Gated Proteasome With Broad Oligopeptide Specificity,” Mol. Microbiol. 59:1405-1416 (2006); Hu et al., “Structure of the Mycobacterium tuberculosis Proteasome and Mechanism of Inhibition by a Peptidyl Boronate,”Mol. Microbiol. 59:1417-1428 (2006)). A peptidyl boronate and an epoxyketone each prevented growth of Mtb and were mycobactericidal during recovery of Mtb from exposure to reactive nitrogen intermediates (Darwin et al., “The Proteasome of Mycobacterium tuberculosis is Required for Resistance to Nitric Oxide,” Science 302:1963-1966 (2003)). Both compounds inhibited a peptidolytic activity in Mtb lysates, while an enantiomer of the peptidyl boronate neither inhibited the peptidolytic activity, prevented growth nor killed Mtb (Darwin et al., “The Proteasome of Mycobacterium tuberculosis is Required for Resistance to Nitric Oxide,” Science 302:1963-1966 (2003)). In vivo silencing of prcBA, the proteasome component genes encoding the β and α subunits, led to a 500-fold decline in viable Mtb in the lungs of mice (Gandotra et al., “In vivo Gene Silencing Identifies the Mycobacterium tuberculosis Proteasome as Essential for the Bacteria to Persist in Mice,” Nat. Med. 13:1515-1520 (2007)).

These observations raised the possibility that proteasome inhibitors might be useful in the treatment of tuberculosis. However, the extensive conservation of proteasome structures militates against species selectivity of proteasome inhibitors. Agents which inhibit the human proteasome might be counterproductive in the treatment of tuberculosis insofar as they might interfere with antigen processing (Yang et al., “The Requirement for Proteasome Activity Class I Major Histocompatibility Complex Antigen Presentation is Dictated by the Length of Preprocessed Antigen,” J. Exp. Med. 183:1545-1552 (1996); and Hughes et al., “The Protease Inhibitor, N-acetyl-Lleucyl-L-leucyl-leucyl-L-norleucinal, Decreases the Pool of Major Histocompatibility Complex Class I-Binding Peptides and Inhibits Peptide Trimming in the Endoplasmic Reticulum,” J. Exp. Med. 183:1569-1578 (1996)), a key part of the host immune response, and given that they exert dose-dependent, mechanism-based host toxicity (Jackson et al., “Bortezomib, a Novel Proteasome Inhibitor, in the Treatment of Hematologic Malignancies,” Cancer Treat. Rev. 31:591-602 (2005)).

The present invention is directed to overcoming these and other deficiencies in the art.

### SUMMARY OF THE INVENTION

One aspect of the present invention relates to a method of treating a subject for a pathogen infection. The method involves administering to the subject a compound of Formula (I).

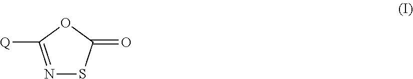

where:

Q is

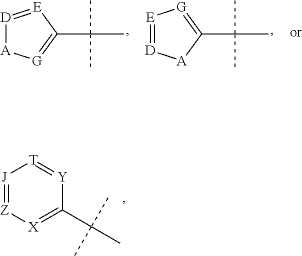

where the crossing dashed line illustrates the bond formed joining Q to the rest of the compound of Formula (I);

A is S or O;

D is N or C, wherein C is bound to R₂;

E is N or C, wherein C is bound to R₁;

G is N or C, wherein C is bound to R₃;

X is N or C, wherein C is bound to R₅;

Y is N or C, wherein C is bound to R₁;

J is N or C, wherein C is bound to R₃;

T is N or C, wherein C is bound to R₂;

Z is N or C, wherein C is bound to R₄;

wherein R₁ to R₅ are independently H, a halogen, —SR₆ —NO₂, —NR₇R₅, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;

R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,

wherein if Q is

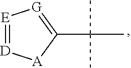

and E and D or E and G are C, wherein R₁ and R₂, or R₁ and R₃, respectively, may form a fused substituted or unsubstituted aromatic or cyclic hydrocarbon ring and if Q is

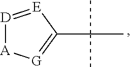

and D and E are C, R₂ and R₁ may form a fused substituted or unsubstituted aromatic ring or cyclic hydrocarbon ring, under conditions effective to treat the subject for a pathogen infection, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₈, —C(O)NHR₁₅, —C(O)NH₂, —CONR₁₆R₁₇, —CHO, —C(O)R₁₈, and —C(O)OR₁₉.

Another aspect of the present invention relates to a method of treating cancer. The method involves administering to the subject a compound of Formula (I).

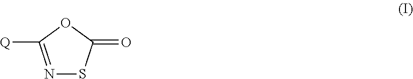

where:

Q is

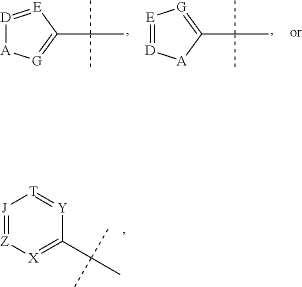

where the crossing dashed line illustrates the bond formed joining Q to the rest of the compound of Formula (I);

A is S or O;

D is N or C, wherein C is bound to R₂;

E is N or C, wherein C is bound to R₁;

G is N or C, wherein C is bound to R₃;

X is N or C, wherein C is bound to R₅;

Y is N or C, wherein C is bound to R₁;

J is N or C, wherein C is bound to R₃;

T is N or C, wherein C is bound to R₂;

Z is N or C, wherein C is bound to R₄;

wherein R₁ to R₅ are independently H, a halogen, —SR₆—NO₂, —NR₇R₃, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted alkynyl, cycloalkyl, haloalkyl, heterocyclyl, or aryl;

R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,

wherein if Q is

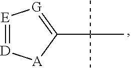

and E and D or E and G are C, R₁ and R₂, or R₁ and R₃, may form a fused substituted or unsubstituted aromatic or cyclic hydrocarbon ring and if Q is

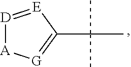

and D and E are C, R₂ and R₁ may form a fused substituted or unsubstituted aromatic ring or cyclic hydrocarbon ring, under conditions effective to treat the subject for cancer, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₃, —C(O)NHR₁₅, —C(O)NH₂, —CONR₁₆R₁₇, —CHO, —C(O)R₁₈, and —C(O)OR₁₉.

Another aspect of the present invention relates to novel compounds of Formula (I):

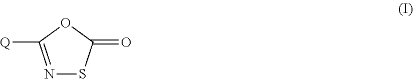

where:

Q is

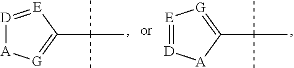

where the crossing dashed line illustrates the bond formed joining Q to the rest of the compound of Formula (I);

A is S or O;

D is N or C, wherein C is bound to R₂;

E is N or C, wherein C is bound to R₁;

G is N or C, wherein C is bound to R₃;

wherein R₁ to R₃ are independently H, a halogen, —SR₆, —NO₂, —NR₇R₈, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted alkynyl, cycloalkyl, haloalkyl, heterocyclyl, or aryl;

R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,

wherein if Q is

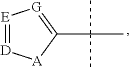

and E and D or E and G are C, R₁ and R₂, or R₁ and R₃, respectively, may form a fused substituted or unsubstituted aromatic or cyclic hydrocarbon ring and if Q is

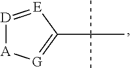

and E and D are C, R₁ and R₂ may form a fused substituted or unsubstituted aromatic or cyclic hydrocarbon ring, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₈, —C(O)NHR₁₅, —C(O)NH₂, —CONR₁₆R₁₇, —CHO, —C(O)R₁₈, and —C(O)OR₁₉;
with the proviso that Q is not an isoxazolyl group, and with the further proviso that at least one of D, E, or G is N if R₁ and R₂, or R₁ and R₃, do not form a fused substituted or unsubstituted aromatic hydrocarbon ring.

The present invention discloses the identification and mechanistic characterization of a novel class of compounds that inhibit Mtb proteasome potently and irreversibly carbonylating the active site Thr1 of the Mtb proteasome.

### BRIEF DESCRIPTION OF THE DRAWINGS

FIGS. 1A-C illustrate structures and modality of inhibition of Mtb PrcAB-OG by oxathiazol-2-ones. FIG. 1A shows various structures of GL1, GL3, GL5, and GL6. FIG. 1B shows GL5, inhibiting the degradation of β-casein by Mtb PrcAB-OG. Mtb PrcAB-OG (12 nM) was incubated with GL5 or GL6 (each 10 μM) at 37° C. for 1 hour prior to addition of β-casein (200 μg/mL). Aliquots were removed immediately or 30, 60, 120 later and degradation of β-casein assessed by SDS-PAGE. PrcΔA denotes the α chain from which the N-terminal octapeptide has been deleted. FIG. 1C illustrates the effect of substrate concentration on inactivation of Mtb PrcAB-OG by GL3 and GL5. Curves are fitted to the equation k_{obs}=k [symbol] /(1+(Kₜ/[I])×(1+[S]/K_{M})) for substrate protection of the enzyme from inactivation (Copeland, R.A., “Enzymes: A Practical Introduction to Structure, Mechanism, and Data Analysis,” pp. 305-349 (2000), which is hereby incorporated by reference in its entirety).

FIGS. 2A-D illustrate kinetic analysis of inactivation of Mtb PrcAB-OG and human proteasomes by oxathiazol-2-ones. FIG. 2A is the graph of progress curves for inhibition of Mtb PrcAB-OG with GL5 at indicated concentrations. FIG. 2B is the plot of pseudo first-order rate constants k_{obs} as function of inhibitor concentration. Values for k_{obs}, derived from the fit of data in FIG. 2A, to equation (1), were plotted against inhibitor concentration. The plot of k_{obs} vs [GL5] yields a straight line crossing the origin, indicating that inactivation is irreversible or very slowly reversible; GL1 and GL3 exhibit a lag at the lower concentrations of inhibitors and approach a maximum at higher concentrations, indicative of positive cooperativity. FIG. 2C is the inhibition of human 20S-β5 by GL5. FIG. 2D is the plot of k_{obs} of GL5/1/3 against h20S as function of inhibition concentration. Values for k_{obs}, derived from the fit of data in FIG. 2C, to equation (1), were plotted against inhibitor concentration. (O) GL5; (□) GL1; (x) GL3.

FIGS. 3A-D illustrate identification of the modified N-terminus of the Mtb proteasome treated with oxathiazol-2-ones. Oxathiazol-2-ones inactivated the Mtb proteasome by carbonylating the γ-OH and α-NH₂ of the active site Thr1. LC-MS/MS was used to identify the modified N-terminus of the Mtb proteasome treated with oxathiazol-2-ones. Mass spectra of tryptic N-terminal heptapeptides from samples that were untreated (FIG. 3A), GL5-treated (FIG. 3B), treated with glutaraldehyde/Na(CN)BH, after trypsin digestion (FIG. 3C), or treated with glutaraldehyde/Na(CN)BH, after GL5 treatment and trypsin digestion (FIG. 3D). All ions were confirmed by MS/MS fragmentation sequencing. The reaction equations illustrate the proposed modification of active site Thr1 by oxathiazol-2-one and the modification of the primary amino groups at both Thr1 and Lys7 with glutaraldehyde and Na(CN)BH₃. Results illustrated for GL5 were identical using GL3.

FIGS. 4A-C illustrate in vivo inactivation of the proteasome in M. bovis BCG by oxathizol-2-ones. FIG. 4A shows the comparison of oxathiazol-2-ones with a peptidyl boronate. BCG (OD [symbol] 0.6-1) was exposed to vehicle DMSO, 50 μM of GL1, GL3 or GL5, or 20 μM MLN-273. After 4 hours, the bacteria were washed twice, lysed mechanically and analyzed for proteasome activity with Ac-YQW-AMC as substrate. FIG. 4B is a graph of time-course for effect of GL5 (50 μM). Experiments were performed as in FIG. 4A except that removal of extracellular compound began at the indicated times. Exposure to GL5 may have continued for up to 15 additional minutes during the washing process. “Untreated” cells were handled in the same manner but without inhibitor or vehicle and were lysed at 60 min. “DMSO” cells received vehicle alone (DMSO, <1% vol/vol) at time 0 and were lysed at 60 min. “T0” cells were treated with inhibitor and then washed immediately. FIG. 4C is the concentration-response for GL5 after 1 hour of exposure. Experiments were also performed as in FIG. 4A except that the concentration of GL5 was varied and lysate was also prepared from an untreated, mutant strain of BCG in which the prcBA genes were selectively disrupted. In the control, cells received vehicle alone. Data are means±SD of triplicates in single experiments, representative of 2 independent experiments.

FIGS. 5A-B illustrate the killing of mycobacteria by oxathiazol-2-ones in synergy with NO. FIG. 5A is a graph of the killing of BCG by GL5, GL1, and GL3 was concentration-dependent and augmented following exposure to a sub-lethal concentration of NO donated by DETA-NO. BCG at an initial number of 2.5×10⁶ CFU/mL (arrow) in Sauton's medium was treated with (open bars) and without (solid bars) DETA-NO (50 μM) and with the indicated oxathiazol-2-one at 10, 25, or 50 μM for four days. Bacteria were then serially diluted in saline and plated for enumeration of surviving CFU. DETA-NO at this concentration prevented replication but caused no killing. FIG. 5B shows the killing of Mtb by GL5 in synergy with NO. Mtb was exposed to 100 μM DETANO (open bars) or 100 μM DETA (solid bars) overnight prior to addition of GL5 at 25 μM or 50 μM or to GL6 (50 μM) as a control. Surviving bacteria were plated 4 days after addition of oxathiazol-2-one. Initial inocula are indicated. DETA-NO at this concentration prevented replication but caused little or no killing. The limit of detection was 4 log₁₀ CFU/ml

FIG. 6 illustrates a mechanism for proteasome inhibition by GL5. Irreversible inhibition of the Mtb proteasome proceeds by the unmarked route.

### DETAILED DESCRIPTION OF THE INVENTION

One aspect of the present invention relates to a method of treating a subject for a pathogen infection. The method involves administering to the subject a compound of Formula (I).

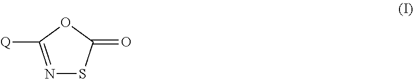

where:

Q is

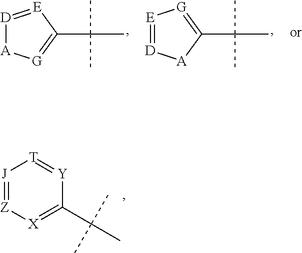

where the crossing dashed line illustrates the bond formed joining Q to the rest of the compound of Formula (I);

A is S or O;

D is N or C, wherein C is bound to R₂;

E is N or C, wherein C is bound to R₁;

G is N or C, wherein C is bound to R₃;

X is N or C, wherein C is bound to R₅;

Y is N or C, wherein C is bound to R₁;

J is N or C, wherein C is bound to R₃;

T is N or C, wherein C is bound to R₂;

Z is N or C, wherein C is bound to R₄;

wherein R₁ to R₅ are independently H, a halogen, —SR₆—NO₂, —NR₇R₆, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;

R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,

wherein if Q is

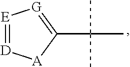

and E and D or E and G are C, R₁ and R₂, or R₁ and R₃, respectively, may form a fused substituted or unsubstituted aromatic or cyclic hydrocarbon ring and if Q is

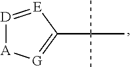

and D and E are C, R₂ and R₁ may form a fused substituted or unsubstituted aromatic ring or cyclic hydrocarbon ring, under conditions effective to treat the subject for a pathogen infection, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₈, —C(O)NHR₁₅, —C(O)NH₂, —CONR₁₆R₁₇, —CHO, —C(O)R₁₈, and —C(O)OR₁₉.

Specific compounds in accordance with the present invention are set forth in Table 1.

TABLE 1
Kinetic parameters of Oxathiazol-2-ones and Their Mycobacterial Killing Effect
α-CT: α -chymotrypsin

| ID | 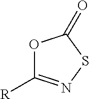 | k_{obs}/[I], M⁻¹s⁻¹ Hu20S Mtb-OG PA28 | | Ratio | BCG killing (25 μM) | | Mtb killing (50 μM) | | IC50 μM (α-CT) |
|---|---|---|---|---|---|---|---|---|---|
| GL5 | 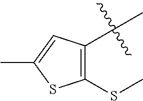 | 376.4 | 0.4 | 1033 | >2 | log | 1.4 | log | 0.064 |
| HT1016 | 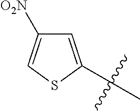 | 731.9 | 12.3 | 59.3 | >3 | log | 2.2 | log | 5.87 |
| HT1041 | 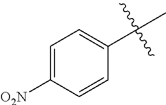 | 597.5 | 12.9 | 46.4 | 0.8 | log | 1.7 | log | 2.08 |
| HT1042 | 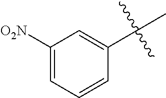 | 729.0 | 10.3 | 71.1 | 2 | log | 1.7 | log | 3.14 |
| HT1043 | 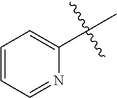 | 460.4 | 0.2 | 2041 | 1 | log | 1.8 | log | 16.6 |
| HT1044 | 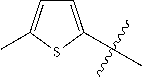 | 1074.6 | 148.4 | 7.2 | 1.7 | log | 2 | log | 8.02 |
| HT1054 | 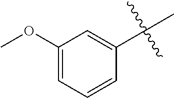 | 636.0 | 14.8 | 43.1 | >3 | log | 2 | log | 2.19 |
| HT1071 | 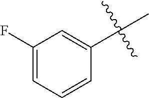 | 1059.3 | 31.3 | 33.8 | >3 | log | 2.2 | log | 2.67 |
| HT1086 | 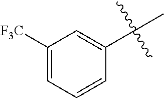 | 154.7 | 0.4 | 356.6 | ND | | ND | | 48.1 |
| HT1113 | 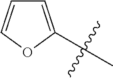 | 434.9 | 10.1 | 43.1 | 1 | log | 2 | log | 22.7 |
| HT1117 | 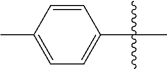 | 432.7 | 53.4 | 8.1 | ND | | ND | | 3.67 |
| HT1118 | 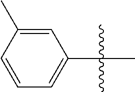 | 132.6 | 11.3 | 11.8 | 0.8 | log | 1.7 | log | 15.8 |

| | | | | | | | |
|---|---|---|---|---|---|---|---|
| HT1146 | 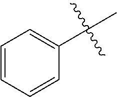 | 408.9 | 31.3 | 13.1 | ND | ND | 10.0 |
| HT1147 | 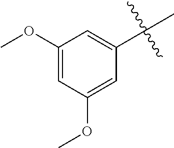 | 45.8 | 0.0 | NA | ND | ND | 83.9 |
| HT1171 | 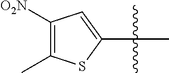 | 3489 | 10.1 | 345 | ND | ND | 18.9 |

The infectious pathogens which can be treated in accordance with this aspect of the present invention include Mycobacterium tuberculosis, Mycobacterium leprae, and other disease-causing Mycobacterium. The diseased subject can be a mammal especially a human.

In one embodiment of the present invention, Q is

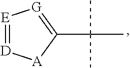

D and E are C, and R₂ and R₁ form a fused substituted or unsubstituted aromatic ring or cyclic hydrocarbon ring, whereby the compound has the following structure of Formula (II):

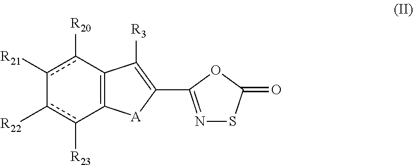

where

[symbol] is independently a single or double bond and

R₂₀ to R₂₃ are independently H, a halogen, —NO₂, —NR₇R₈, —CONR₁₆R₁₇, substituted or unsubstituted C₁-C₂₀ alkyl, substituted or unsubstituted C₁-C₂₀ alkenyl, substituted or unsubstituted C₁-C₂₀ alkynyl, cycloalkyl, heterocyclyl, or aryl.

Examples of compounds of Formula (II) are

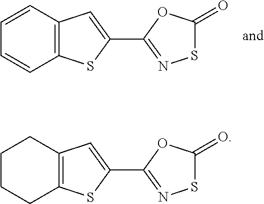

In another embodiment of the present invention, Q is

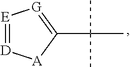

E and G are C, and R₁ and R₃ form a fused substituted or unsubstituted aromatic ring or cyclic hydrocarbon ring whereby the compound has the following structure of Formula (III):

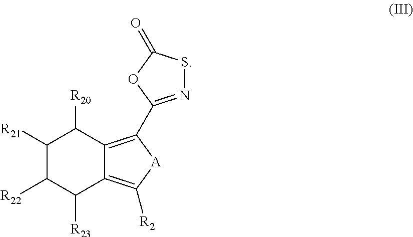

In another embodiment of the present invention, Q is

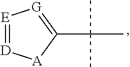

and the compound of Formula (I) has the structure of Formula (IV)

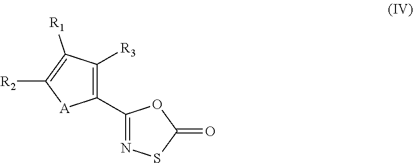

Examples of compounds of Formula (IV) are

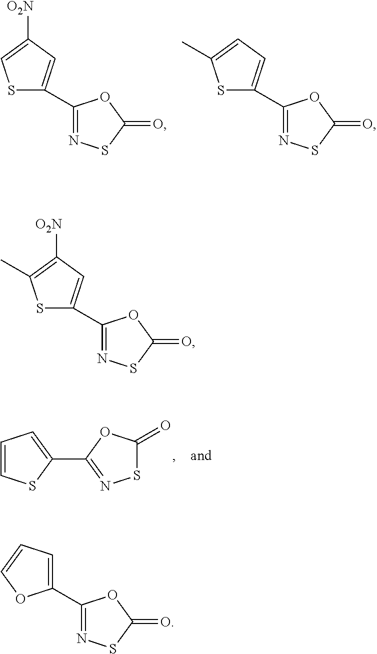

In one embodiment of the present invention, Q is

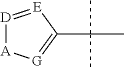

and the compound of Formula (I) has the structure of Formula (V)

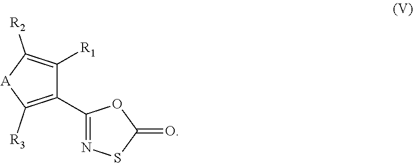

Examples, of the compounds of Formula (V) are

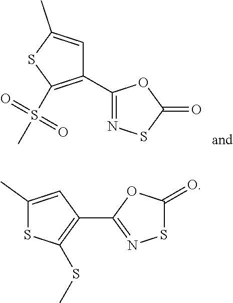

In one embodiment of the present invention, Q is

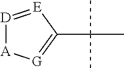

and the compound of Formula (I) is selected from the group consisting of

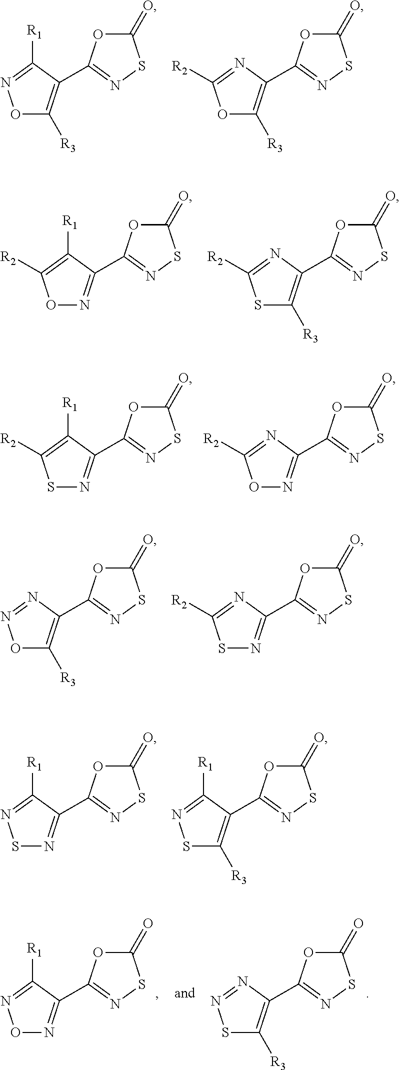

In another embodiment of the present invention, Q is

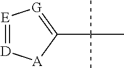

and the compound of Formula (I) is selected from the group consisting of

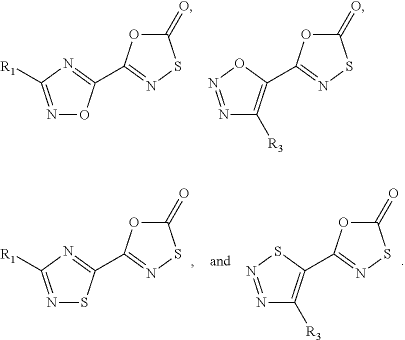

In yet another embodiment of the present invention, Q is

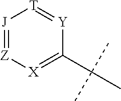

and the compound of Formula (I) has the structure of Formula (VI)

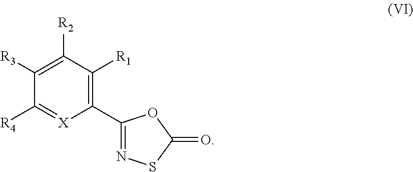

Examples of the compounds of Formula (VI) are 5-phenyl-1,3,4-oxathiazol-2-one, 5-(4-nitrophenyl)-1,3,4-oxathiazol-2-one, 5-(3-nitrophenyl)-1,3,4-oxathiazol-2-one, 5-(2-pyridinyl)-1,3,4-oxathiazol-2-one, 5-(3-methoxyphenyl)-1,3,4-oxathiazol-2-one, 5-(3-fluorophenyl)-1,3,4-oxathiazol-2-one, 5-(3-(trifluoromethyl)-phenyl)-1,3,4-oxathiazol-2-one, 5-(4-tolyl)-1,3,4-oxathiazol-2-one, 5-(3-tolyl)-1,3,4-oxathiazol-2-one, and 5-(3,5-dimethoxyphenyl)-1,3,4-oxathiazol-2-one.

In another embodiment of the present invention, Q is

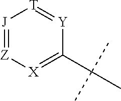

and the compound of Formula (I) is selected from the group consisting of

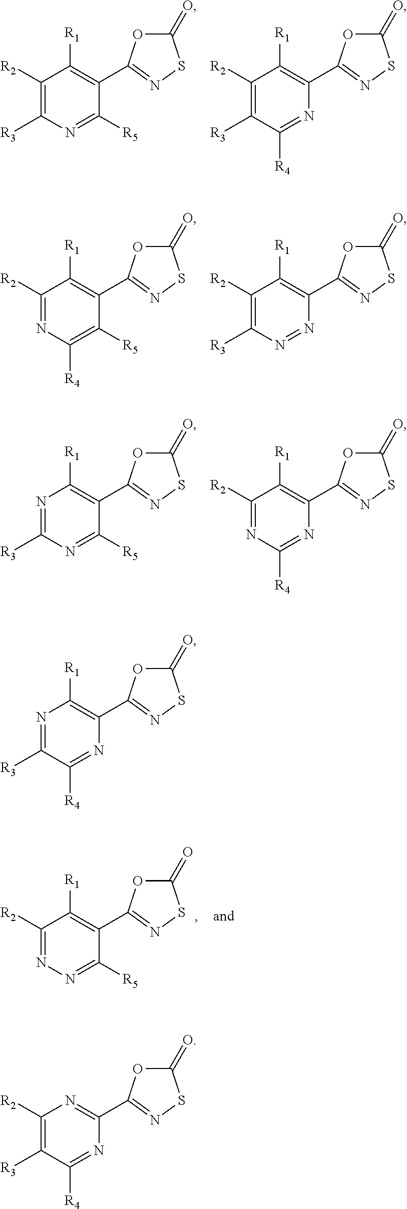

Another aspect of the present invention relates to a method of treating cancer. The method involves administering to the subject a compound of Formula (I).

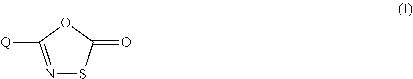

where:

Q is

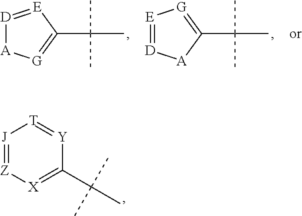

where the crossing dashed line illustrates the bond formed joining Q to the rest of the compound of Formula (I);

A is S or O;

D is N or C, wherein C is bound to R₂;

E is N or C, wherein C is bound to R₁;

G is N or C, wherein C is bound to R₃;

X is N or C, wherein C is bound to R₅;

Y is N or C, wherein C is bound to R₁;

J is N or C, wherein C is bound to R₃;

T is N or C, wherein C is bound to R₂;

Z is N or C, wherein C is bound to R₄;

wherein R₁ to R₅ are independently H, a halogen, —SR₅—NO₂, —NR₇R₈, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;

R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,

wherein if Q is

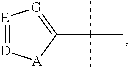

and E and D or E and G are C, R₁ and R₂, or R₁ and R₃, respectively, may form a fused substituted or unsubstituted aromatic or cyclic hydrocarbon ring and if Q is

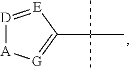

and D and E are C, R₂ and R₁ form a fused substituted or unsubstituted aromatic ring or cyclic hydrocarbon ring, under conditions effective to treat the subject for a pathogen infection, wherein the substituted alkyl, alkenyl, alkynyl, aromatic, or cyclic hydrocarbon ring groups have substituents selected from the group consisting of hydroxyl, halogen, —CN, —NO₂, —SR₆, —SO₂R₉, —NH₂, —NHR₁₃, —NHSO₂R₁₄, —NR₇R₈, —C(O)NHR₁₅, —C(O)NH₂, —CONR₁₆R₁₇, —CHO, —C(O)R₁₈, and —C(O)OR₁₉.

In treating cancer, the same compounds are used as described above for treating pathogen infection.

Forms of cancer which can be treated in accordance with the present invention include carcinoma, sarcoma, lymphoma, and myeloma.

In practicing the method of the present invention, agents suitable for treating a subject can be administered using any method standard in the art. The agents, in their appropriate delivery form, can be administered orally, intradermally, intramuscularly, intraperitoneally, intravenously, subcutaneously, or intranasally. The compositions of the present invention may be administered alone or with suitable pharmaceutical carriers, and can be in solid or liquid form, such as tablets, capsules, powders, solutions, suspensions, or emulsions.

The agents of the present invention may be orally administered, for example, with an inert diluent, or with an assimilable edible carrier, or it may be enclosed in hard or soft shell capsules, or it may be compressed into tablets, or they may be incorporated directly with the food of the diet. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. (Abuchowski and Davis, “Soluble Polymer-Enzyme Adducts,” In: Enzymes as Drugs, Hocenberg and Roberts, eds., Wiley-Interscience, New York, N.Y., pp. 367-383 (1981), which are hereby incorporated by reference in their entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt

The agents of the present invention may also be administered directly to the airways in the form of an aerosol. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

Effective doses of the compositions of the present invention, for the treatment of cancer or pathogen infection vary depending upon many different factors, including type and stage of cancer or the type of pathogen infection, means of administration, target site, physiological state of the patient, other medications or therapies administered, and physical state of the patient relative to other medical complications. Treatment dosages need to be titrated to optimize safety and efficacy.

Another aspect of the present invention relates to the compound of Formula (I):

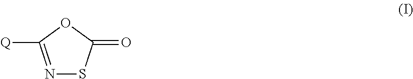

where:

Q is

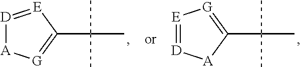

where the crossing dashed line illustrates the bond formed joining Q to the rest of the compound of Formula (I);

A is S or O;

D is N or C, wherein C is bound to R₂;

E is N or C, wherein C is bound to R₁;

G is N or C, wherein C is bound to R₃;

wherein R₁ to R₃ are independently H, a halogen, —SR₆—NO₂, —NR₇R₈, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;

R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,

wherein if Q is

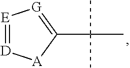

and E and D or E and G are C, R₁ and R₂, or R₁ and R₃, respectively, may form a fused substituted or unsubstituted aromatic or cyclic hydrocarbon ring and if Q is

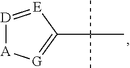

and D and E are C, R₂ and R₁ may form a fused substituted or unsubstituted aromatic or cyclic hydrocarbon ring, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₅, —C(O)NHR₁₅, —C(O)NH₂, —CONR₁₆R₁₇, —CHO, —C(O)R₁₈, and —C(O)OR₁₉;
with the proviso that Q is not an isoxazolyl group, and with the further proviso that at least one of D, E, or G is N if R₁ and R₂, or R₁ and R₃, do not form a fused substituted or unsubstituted aromatic hydrocarbon ring.

To the extent within the scope of the compound recited in the preceding paragraph, that compound can take the form of the sub-classes of compounds and specific compounds described above

In the embodiment of the present invention, where Q is

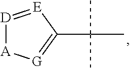

suitable compounds of Formula (I) have the following structures:

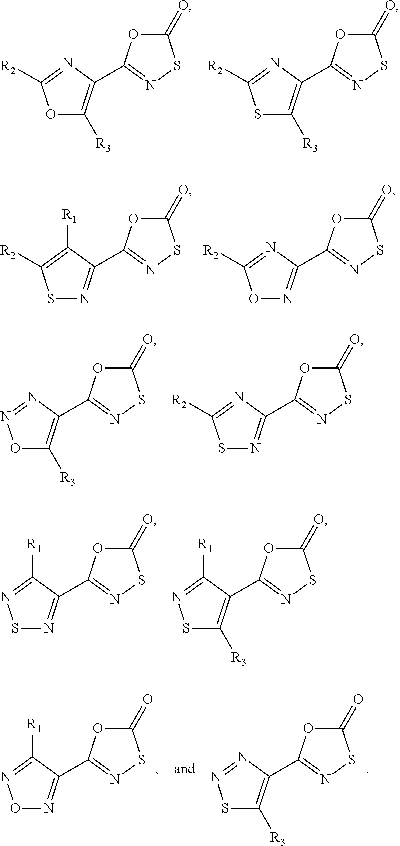

### EXAMPLES

The Examples set forth below are for illustrative purposes only and are not intended to limit, in any way, the scope of the present invention.

### Example 1

### Materials

The “open gate” mutant of recombinant Mtb proteasome (PrcAB-OG) was over-expressed in E. coli and purified as reported (Lin et al., “Mycobacterium tuberculosis prcBA Genes Encode a Gated Proteasome With Broad Oligopeptide Specificity,” Mol. Microbiol. 59:1405-1416 (2006), which is hereby incorporated by reference in its entirety). Human red blood cell 20S and PA28 were purchased from Boston Biochem (Cambridge, Mass.). GL1, GL2, GL3, GL4 were purchased from TimTec LLC (DE, USA) and GL5, GL6, GL7 from ChemDiv, Inc. (CA, USA). Bortezomib was purchased from LC Laboratories (MA, USA). Substrates Ac-RFW-AMC, Ac-YQW-AMC were synthesized by AnaSpec (CA, USA) and used for detailed kinetic analyses. Different substrates were chosen according to the nature of the experiments.

### Example 2

### High Throughput Screen

The screening was conducted with compounds from ChemDiv, Chembridge, Spectrum, Preswick, and Cerep.

### Example 3

### Kinetics

Kinetic measurements were made on a Hitachi F-2500 fluorescence spectrophotometer with 0.238 nM PrcAB-OG in 20 mM HEPES, 0.5 mM EDTA, pH 7.5, 0.1 mg/mL BSA and 25 μM Ac-RFWAMC for Mtb PrcAB-OG or 25 μM Suc-LLVY-AMC for human 20S at 37° C. After steady state conditions were achieved, inhibitors were added and substrate cleavage was monitored (λₑₓ=360 nm, λₑₓ=460 nm) at 5-second intervals for 60 minutes or until no activity remained. The data were fitted to equation (Corbett et al., “The Growing Burden of Tuberculosis: Global Trends and Interactions with the HIV Epidemic,” Arch. Intern. Med. 163:1009-1021 (2003), which is hereby incorporated by reference in its entirety) to determine k_{obs} [symbol] using Prism (GraphPad Software, Inc. San Diego, Calif.).

### Example 4

### LC-MS/MS Analysis

Mtb PrcAB-OG (415 μg/mL; 8.2 μM active sites) in 20 mM HEPES, 0.5 mM EDTA, pH 7.5, was incubated with 500 μM GL5 or GL3 at room temperature until the activity assay demonstrated that inactivation was complete. A control sample was incubated for the same time with an equivalent volume of DMSO. The samples were then run on SDS-page to separate α and β subunits. The gel bands corresponding to untreated and inhibitor-treated PrcB were excised from the gel, reduced with 10 mM DTT, alkylated with 55 mM iodoacetamide and digested with sequence grade modified trypsin (Promega) in ammonium bicarbonate buffer at 37° overnight. The digestion products were analyzed by LC-MS/MS and LC-MS with Thermo LTQ Orbitrap and Applied Biosystems QSTAR mass spectrometers, respectively. One tenth of the digestion products for each sample were also analyzed by MALDI-TOF with a PerSeptive MALDI-TOF DE-STR mass spectrometer. For LC-MS/MS analysis, each digestion product was separated by gradient elution with a Dionex capillary/nano-HPLC system that is directly interfaced with the mass spectrometer. MS/MS data were searched using the MASCOT search engine for identifying proteins and modifications. For in-gel modification of primary amine groups, gel slices of untreated- and treated-proteins were incubated with 500 mM sodium cyanoborohydride and 2.5% glutaraldehyde at 37° C. for the desired time. Reactions were stopped by addition of 1M Tris-HCl.

### Example 5

### Inhibition of Proteasome Activity in Mycobacteria

20-ml cultures of BCG in Sauton's medium at optical density 0.8-1.0 at 580 nm were treated with GL1, GL3, or GL5 at 50 μM or with MLN-273 (Millennium Pharmaceuticals Inc.) at 20 μM. After 4 hours, BCG cells were harvested by centrifugation at 3,000 g for 10 minutes. Pellets were washed with PBS with 0.02% Tween 80 and again with PBS lacking Tween. Pellets were resuspended in assay buffer (20 mM HEPES, 0.5 mM EDTA, pH 7.4, 100 μM phenylmethylsulfonyl fluoride (PMSF)), and lysed by mechanical beating with Zirconium beads. Lysates were spun at 16,000 g for 10 minutes and protein concentrations of the supernatants determined by the Bradford assay. Concentration studies used GL5 for 1 hour. Activity assays were performed with ˜10 μg of lysate protein using Ac-YQW-AMC (50 μM) as substrate in 20 mM HEPES, 0.5 mM EDTA, pH 7.5, 100 μM PMSF, 0.02% SDS, 0.1 mg/mL BSA at 37° C.

### Example 6

### Mycobactericidal Activity

M. bovis BCG (ATCC 35734) and Mtb H37Rv (ATCC 25618) were cultivated in Sauton's medium pH 7.4 with 0.4% L-asparagine, 0.2% glycerol and 0.02% Tween 80. Mid-log phase cultures (A₅₆₀ [symbol] 0.8-1.0) were diluted to 0.05-0.1 (A₅₈₀) and quantified by CFU. Mycobacteria were incubated under indicated conditions in 96 well plates in 200 μl, then serially diluted in PBS with 0.02% Tween 80, pH 7.2 and plated for CFU on Middlebrook 7H11 agar plates with 10% Middlebrook OADC enrichment.

### Example 7

### Identification of Oxathiazol-2-Ones as Proteasome Inhibitors

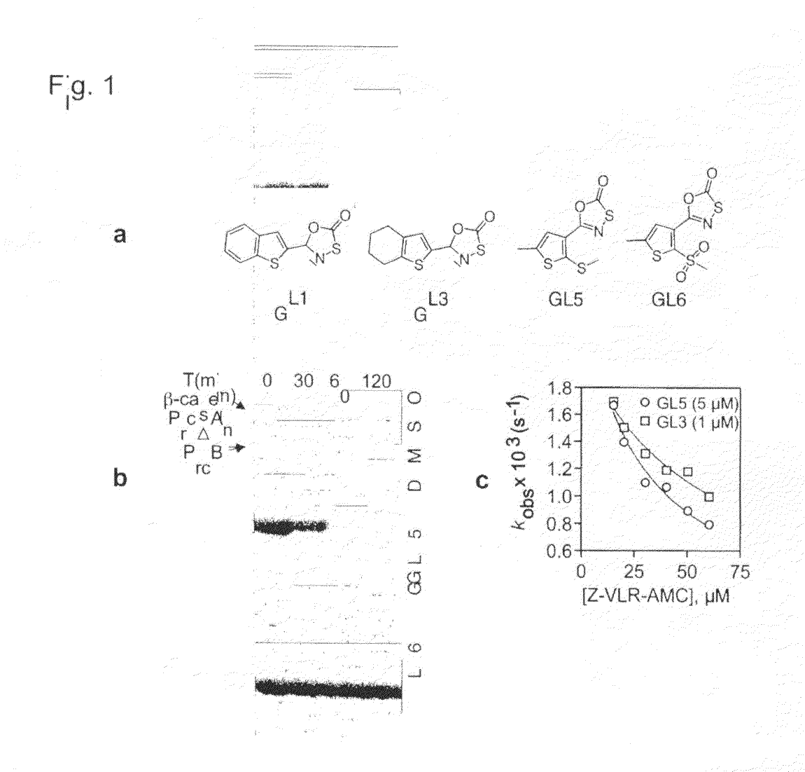

Mtb PrcAB-OG was screened with Suc-LLVY-7-amido-4-methylcoumarin (AMC) as substrate, recording the fluorescence of AMC released upon cleavage. From 20,000 commercially available compounds, a potent inhibitor 5-(5-methyl-2-(methylthio)thiophen-3-yl)-1,3,4-oxathiazol-2-one was identified, here termed GL5 (FIG. 1A). From commercial sources, was assembled a total of seven 1,3,4-oxathiazol-2-ones for further analysis (FIG. 1A). In preliminary studies with oligopeptide substrates, it was determined that IC₅₀'s of each oxathiazol-2-one except GL6 against the Mtb proteasome were far lower than against the human proteasome. GL6, the sulfone analog of GL5, inhibited neither. At 10 μM, GL5, but not GL6, prevented the Mtb proteasome from degrading an intact protein, β-casein (FIG. 1B). Thus, the inhibitory activity of oxathiazol-2-ones is not limited to the action of the proteasome against small peptides, and is strongly influenced by minor changes in oxathiazol-2-one structure.

### Example 8

### Mode of Inhibition

Inhibition of the Mtb proteasome by oxathiazol-2-ones was irreversible by several criteria. First, no activity was recovered by diluting pre-incubated Mtb proteasome with GL1 633-fold into substrate-containing reaction mixture. Second, dialysis of the Mtb proteasome treated with GL3, GL5, bortezomib (a reversible inhibitor) (Williamson et al., “Comparison of Biochemical and Biological Effects of ML858 (Salinosporamide A) and Bortezomib,” Mol. Cancer Ther. 5:3052-3061 (2006), which is hereby incorporated by reference in its entirety) or NPI-0052 (an irreversible inhibitor) (Groll et al., “Crystal Structures of Salinosporamide A (NPI-0052) and B (NPI-0047) in Complex With the 20S Proteasome Reveal Important Consequences of Beta-Lactone Ring Opening and a Mechanism for Irreversible Binding,” J. Am. Chem. Soc. 128:5136-5141 (2006), which is hereby incorporated by reference in its entirety), for 18 hours against inhibitor-free buffer, did not restore the activity of proteasomes treated with GL3, GL5 or NPI-0052, while 70% of activity was recovered in the bortezomib-treated sample. These results indicate that oxathiazol-2-ones are either irreversible or practically irreversible inhibitors of the Mtb proteasome. However, within the limit of solubility of the oxathiazol-2-ones in aqueous buffer, inhibition of the human proteasome was too limited to make reliable measurements in similar experiments. Thus, kinetic analyses were investigated to compare the effects of oxathiazol-2-ones on proteasomes of the two species.

First, irreversible inhibitors can be competitive, noncompetitive, or uncompetitive with respect to substrate (Copeland, R.A., “Evaluation of Enzyme Inhibitors in Drug Discovery: A Guide for Medicinal Chemists and Pharmacologists,” pp. 214-248 (2005), which is hereby incorporated by reference in its entirety). Accordingly, the effect of the concentration of substrate (Z-VLR-AMC) on the rate of inactivation of the Mtb proteasome by a fixed concentration of inhibitor (5 μM for GL5 and 1 μM for GL3) was tested. Increasing substrate concentration reduced the rate of proteasome inactivation (FIG. 1C). Thus, the oxathiazol-2-ones appear to compete with substrate at the active site of the Mtb proteasome.

### Example 9

### Kinetics of Proteasome Inhibition by Oxathiazol-2-Ones

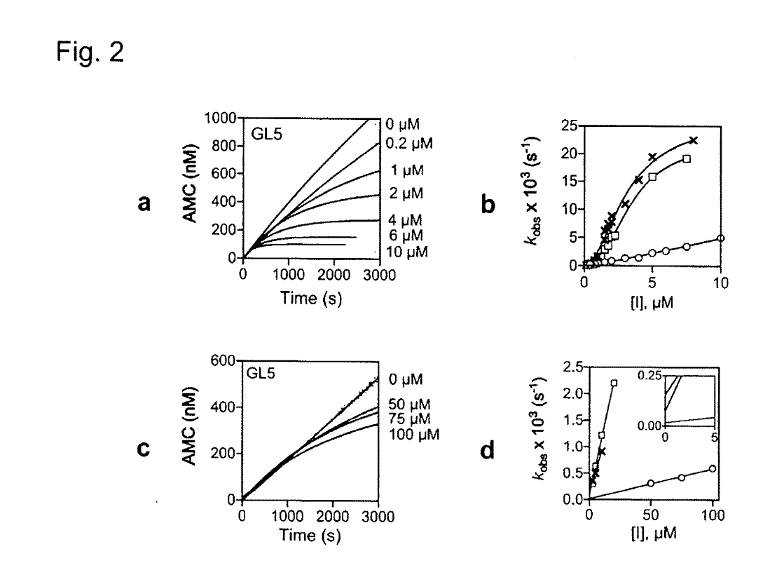

Kinetic analysis provided further evidence of irreversible inhibition of the Mtb proteasome and showed that inhibition of the human proteasome, by contrast, was reversible. Assessment of reaction progress curves in the presence of various concentrations of GL5 revealed a time-dependent conversion from an initial state to a steady-state velocity of zero, characteristic of slow-binding inhibition kinetics due to irreversible enzyme inactivation (FIG. 2A). The progress curves were fitted by a nonlinear least-squares method to equation (1), where P is the product, v₀ is the initial velocity, v [symbol] is the steady-state velocity (in the case of irreversible inhibition, v, equals to zero), k_{obs} [symbol] is the apparent first order rate constant for conversion from the initial velocity phase to the steady-state velocity phase, and t is time (Copeland, R.A., “Enzymes: A Practical Introduction to Structure, Mechanism, and Data Analysis,” pp. 305-349 (2000), which is hereby incorporated by reference in its entirety).

[P]=v,t+[(v₀−v [symbol] )/k_{obs} [symbol] ]×[1−exp(−k_{obs} [symbol] t)] Equation (1)

As shown in FIG. 2B, a linear dependence of k_{obs} [symbol] on GL5 concentration [I] was observed that intersected at the origin, indicative of one-step irreversible or very slowly reversible inhibition (Copeland, R.A., “Evaluation of Enzyme Inhibitors in Drug Discovery: A Guide for Medicinal Chemists and Pharmacologists,” pp. 214-248 (2005), which is hereby incorporated by reference in its entirety). The slope gave an apparent value of k_{obs} [symbol] /[I] 476±9.3 M⁻¹ s⁻¹, also referred to as k [symbol] /K [symbol] for an irreversible inhibitor, which was then corrected by correcting K [symbol] by equation K [symbol] =K^{app}/(1+[S]/K_{M}) to remove the effect of substrate competition (Copeland, R.A., “Enzymes: A Practical Introduction to Structure, Mechanism, and Data Analysis,” pp. 305-349 (2000), which is hereby incorporated by reference in its entirety). Hence, the k [symbol] /K [symbol] for GL5 vs Mtb proteasome was 624±12.2 M⁻¹ s¹ (Table 2).

TABLE 2
Kinetic parameters of oxathiazol-2-ones

| | | | | | | Hill coefficient |
|---|---|---|---|---|---|---|
| | Mtb PrcAB-OG | | | H20S-β5 | | (Mtb |
| | k_{inact} (s⁻¹)^{a} | K [symbol] (μM)^{a} | k_{inact}/K [symbol] (M⁻¹s⁻¹) | k_{obs}/[I]^{c} (M⁻¹s⁻¹) | Selectivity^{d} | PrcAB-OG) |
| GL1 | 0.018 ± 0.001 | 14.4 ± 1.0 | 1250 ± 111^{b} | 194 ± 5.5 | 6.4 | 2.3 |
| GL3 | 0.020 ± 0.002 | 6.9 ± 0.7 | 2898 ± 413^{b} | 128 ± 12.3 | 22.6 | 1.9 |
| GL5 | N/A^{a} | N/A^{a} | 625 ± 12.2^{b} | 9.8 ± 1.3 | 63.8 | 1 |

^{a}k_{inact}: the maximum rate of inactivation achieved at infinite concentration of inactivator. K [symbol] : the concentration of inactivator that yields a rate of inactivation equal to ½ k_{inact}.
^{b}Error shown is the propagated error from k_{inact} and K [symbol] .
^{c}For Mtb proteasome, k_{obs}/[I] equals k_{inact}/K [symbol] . All inactivation constants were converted from apparent values by using equation: K = K^{app}/(1 + [S]/K_{M})
^{d}The ratio of k_{inact}/K [symbol] _{(Mtb-20S OG)}versus k_{obs}/[I]_{(H20S} _{β5)}. [symbol]
indicates data missing or illegible when filed

GL1 and GL3 also exhibited time-dependent inhibition of the Mtb proteasome, but their kinetics appeared to be more complex, in that the plots of k_{obs} [symbol] vs [I] showed a lag at lower concentrations of inhibitor, reaching saturation at higher concentrations (FIG. 2B). For a time-dependent irreversible inhibitor, the dependence of k_{obs} [symbol] inhibitor concentration can be described by a rectangular hyperbola (equation (3)) (Kitz et al., “Esters of Methanesulfonic Acid as Irreversible Inhibitors of Acetylcholinesterase,” J. Biol. Chem. 237:3245-3249 (1962), which is hereby incorporated by reference in its entirety), from which can be extracted the values of k [symbol] , the maximal rate constant for inactivation of the enzyme by the inhibitor, and Kₜ, the concentration that yields a rate of inactivation half of k [symbol] . The lag at lower concentrations may be indicative of cooperativity, in which the initial binding of inhibitor accelerates subsequent inactivation. The mechanistic and kinetic origins and significance of this behavior on the part of GL1 and GL3 remain to be determined. Equation (2) was modified to take the “apparent cooperativity” into account, yielding equation (3), where h is the Hill coefficient.

k=k_{obs} [symbol] /(1+(K [symbol] /[I]) Equation (2)

k_{obs} [symbol] =k [symbol] /(1+K [symbol] /[I]^{h}) Equation (3)

This analysis yielded the inhibitory kinetic values shown for GL1 and GL3 in Table 2. The Hill coefficients (h) of 2.3 and 1.9 might imply that binding of GL1 or GL3 to half of the available active sites of the Mtb proteasome accelerates the inactivation of the whole enzyme complex.

As noted, the cytotoxicity of proteasome inhibitors is correlated with their inhibition of the proteasome's chymotryptic-like activity (Adams et al., “Proteasome Inhibitors: A Novel Class of Potent and Effective Antitumor Agents,” Cancer Res. 59:2615-2622 (1999), which is hereby incorporated by reference in its entirety). Accordingly, the next focus was placed on the chymotryptic-like site of the β5 subunit of proteasomes isolated from human erythrocytes in the presence of the human proteasome activator PA28 and obtained the k_{obs} [symbol] /[1] values for GL5 (tested over the range 50-100 μM, FIG. 2C), GL1 (2.5-20 μM), and GL3 (2.5-10 μM). The plots of k_{obs} [symbol] vs [I] for all three compounds yielded straight lines, indicating a simple one-step inactivation mechanism (Copeland, R.A., “Enzymes: A Practical Introduction to Structure, Mechanism, and Data Analysis,” pp. 305-349 (2000), which is hereby incorporated by reference in its entirety). Surprisingly, none of the plots passed through the origin (FIG. 2D), indicating that inactivation of the human proteasome β5 subunit by these three oxathiazol-2-ones is reversible.

Species selectivity of the inhibitors was characterized kinetically by comparing the second-order rate constants of k [symbol] /K [symbol] for the Mtb proteasome and k_{obs} [symbol] /[I] for the β5 subunit of the human proteasome. The oxathiazol-2-ones tested ranged from 6- to 64-fold more potent against the Mtb proteasome than the human β5 subunit (Table 2).

### Example 10

### Identification of the Covalent Adduct of Mtb Proteasome β Subunit with Oxathiazol-2-Ones

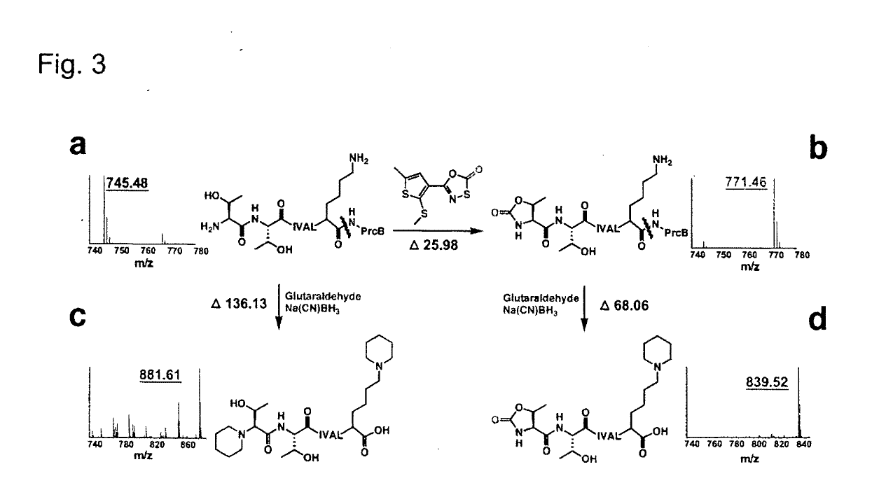

The apparent irreversible and competitive manner of inhibition suggested that the oxathiazol-2-ones probably inactivate the Mtb proteasome by covalently bonding with the active site Thr1. To test this hypothesis, Mtb proteasomes that had been treated or not with oxathiazol-2-ones GL3 or GL5 were trypsinized and LC-MS/MS was used to identify peptides from the β subunits. Peptides identified by LC-MS/MS collectively covered over 98% of protein sequence. One peptide ion, which had m/z 771.46, was unique to the GL5-treated Mtb proteasome β subunit (FIG. 3B); the same ion was also unique to the GL3-treated Mtb proteasome β subunit. The mass of this peptide was 26 Da higher than that of the N-terminal heptapeptide (TTIVALK (SEQ ID NO:1)) with MW 745.48 (M+1)¹⁺ that was identified only in the untreated samples (FIG. 3A), indicative of addition of a carbonyl at the expense of two hydrogen ions. The ion at m/z 771.46 was subjected to LC-MS/MS analysis. Its fragmentation pattern confirmed that the altered species corresponded to the N-terminal heptapeptide and indicated that the modification must occur at the N-terminal Thr1 and/or Thr2.

To further investigate the modification site, the ability to modify primary amino groups by reaction with glutaraldehyde and sodium cyanoborohydride was exploited, resulting in a mass increase of 68.06 Da for each such modification. Modifying the Mtb proteasome with glutaraldehyde and sodium cyanoborohydride without prior exposure to an oxathiazol-2-one led to identification of the N-terminal heptapeptide (TTIVALK (SEQ ID NO:1)) with MW 881.48 (M+H)¹⁺, a mass increase of 136.13 Da, consistent with modification of the primary amino groups of Thr1 and Lys7 (FIG. 3C), as confirmed by the fragmentation pattern in MS/MS. In contrast, only one glutaraldehyde-dependent modification of the N-terminal heptapeptide was detected in proteasomes that had first been treated with GLS. The corresponding ion had m/z 839.52 (M+H)¹⁺, representing a mass increase of 68.06 Da (FIG. 3D). The pattern of the fragmentation in MS/MS confirmed that only the Lys7 side chain had been modified. Again, results with GL3 in place of GL5 were identical.

It was deduced that the 26-Da increase in MW resulted from carbonylation of NH₂ and OH groups of the active site or the adjacent amino acid side chains in the N-terminal heptapeptide. The heptapeptide offers three possible carbonylation sites: 1) the α-NH₂ and γ-OH groups of Thr1; 2) the α-NH₂ group of the Thr1 and the γ-OH group of Thr2; 3) the amide NH group of Thr2 and the γ-OH group of Thr1. Because the γ-OH group is the nucleophile of the active site Thr1, which initiates a nucleophilic attack on carbonyl groups of substrates or oxathiazol-2-ones, the second possibility can be excluded. Given that only the NH₂ group of Lys7 was modified by glutaraldehyde/Na(CN)BH₂ in oxathiazol-2-onetreated proteasomes, the third possibility can be ruled out; otherwise the glutaraldehyde/Na(CN)BH, modification would have yielded a 162 Da mass shift for the peptide. All these lines of evidence suggest that the N-terminal α-NH, and γ-OH groups of Thr1 of the β chain of the Mtb proteasome participate in a covalent reaction with both GL3 and GL5 oxathiazol-2-ones to form the oxazolidin-2-one species.

### Example 11

### Impact of Oxathiazol-2-Ones on Mycobacteria

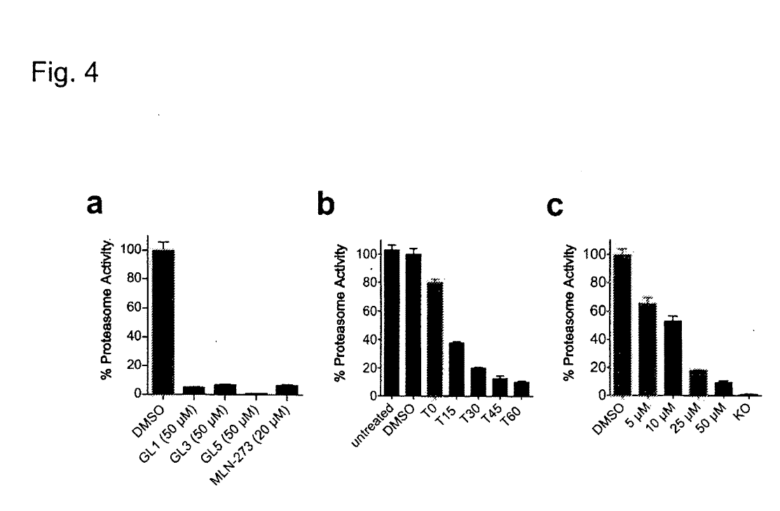

The next step was to examine whether oxathiazol-2-ones could penetrate the mycobacterial cell wall and inhibit native proteasomes in the cytosolic milieu. Mycobacterium bovis var. BCG was then incubated with GL1, GL3, or GL5 (each at 50 μM) for 4 hours, the cells were washed to remove extracellular compound, lysed, and the residual proteasome activity was measured. The peptidyl boronate MLN-273 (N-morpholino-(L)-naphthylAla-(L)-Leu-boronic acid) (25 μM) served as a positive control. Like MLN-273, the oxathiazol-2-ones each blocked >90% of proteasome activity, approaching the level of inhibition achieved by disrupting the prcBA genes (FIGS. 4A and C). Time course and concentration-response studies (FIGS. 4B and C) showed that intrabacterial proteasomes were inhibited ≧50% after 30 minutes of exposure to 50 μM GL5, ≧50% after 60 minutes of exposure to 10 μM GL5, and by 90% after 60 minutes exposure to 50 μM GL5. Thus, the oxathiazol-2-ones enter BCG rapidly and inhibit native proteasomes extensively.

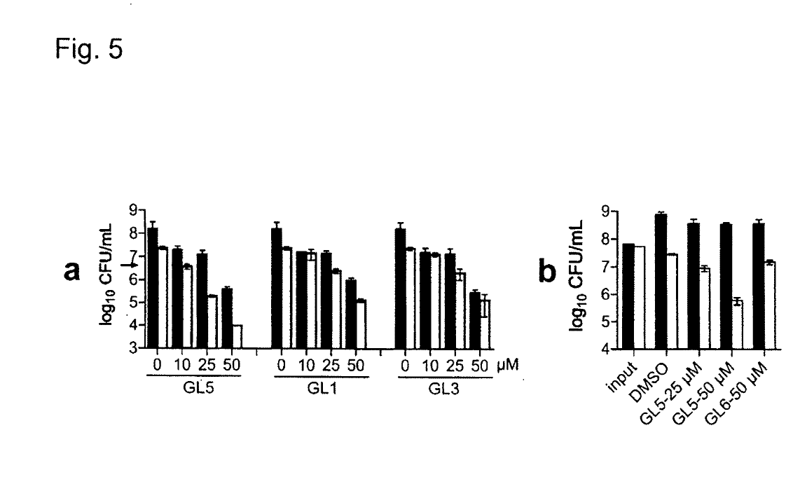

The inhibitors were investigated to ascertain that they were mycobactericidal alone and during recovery of mycobacteria from exposure to NO. Provision of NO in vitro was intended to mimic the nitroxidative stress that limits Mtb's replication in wild type mice, as judged by the profound acceleration of Mtb replication in mice whose alleles for inducible nitric oxide synthase were disrupted (MacMicking et al., “Identification of Nitric Oxide Synthase as a Protective Locus Against Tuberculosis,” Proc. Natl. Acad. Sci. USA 94:5243-5248 (1997), which is hereby incorporated by reference in its entirety). NO was provided from the decomposition of 2,2-(hydroxynitrosohydrazino)-bis-ethanamine (DETA-NO) at 50 μM. By itself, DETA-NO at this concentration did not affect bacterial viability. Although DETA-NO decomposes with a t [symbol] of 20 hours at 37° C. and pH 7.0, it was added only at the outset of the 4-day experiment. The oxathiazol-2-ones proved to be bacteriocidal to BCG both alone and in synergy with DETA-NO. GL1, GL3, and GL5 were each able to kill BCG. GL5 was the most effective, reducing the number of colony forming units (CFU) by 400-fold at 25 μM and by more than 2000-fold at 50 μM when the cells had been exposed to DETA-NO (FIG. 5A).

An additional investigation on GL5 ability to kill Mtb was performed. Mtb were exposed overnight to 100 μM DETA-NO or 100 μM DETA, the product that remained after DETA-NO decomposed, followed by incubation with GL5 or its inactive sulfone, GL6. Within 4 days, GL5 (50 μM) reduced the number of CFU by 47-fold if the Mtb had been exposed to DETA-NO but not if they had been exposed to DETA. GL6 did not lead to a reduction of CFU in either condition (FIG. 5B). Thus, only the oxathiazol-2-one that inhibited Mtb's PrcAB could kill Mtb, and only in conjunction with exposure to sublethal nitric oxide.

### Example 12

### Impact of Oxathiazol-2-Ones on Mammalian Cells

Given that proteasome-inhibiting oxathiazol-2ones killed mycobacteria, their impact on the survival of mammalian cells was tested. GL5 and GL6 showed no evident cytotoxicity when incubated with human skin fibroblasts, human lung fibroblasts, Vero monkey kidney epithelial cells, or mouse macrophage-like RAW264.7 cells at concentrations up to 50 μM for 4 days. In contrast, less than 10% of Vero cells remained viable after 4-day exposure to 25 nM bortezomib. Finally, because most Mtb in nature resides in human macrophages, we exposed human monocyte-derived macrophages (mostly multinucleated giant cells) to 100 μM GL3, GL5 or GL6 for 7 days. There was no morphologic sign of cytotoxicity. Again, in contrast, some of the human macrophages exposed to as little as 0.5 μM bortezomib for 7 days rounded up or appeared to disintegrate.

Proteasomes play critical roles in eukaryotes (Ciechanover, A., “Proteolysis: From the Lysosome to Ubiquitin and the Proteasome,” Nat. Rev. Mol. Cell. Biol. 6:79-87 (2005); and Goldberg, A. L., “Functions of the Proteasome: From Protein Degradation and Immune Surveillance to Cancer Therapy,” Biochem. Soc. Trans. 35:12-17 (2007), which are hereby incorporated by reference in their entirety). As the major means of protein turnover, they help cells adapt to changing circumstances, dispose of oxidized (Jung et al., “Oxidized Proteins: Intracellular Distribution and Recognition by the Proteasome,” Arch. Biochem. Biophys. 462:231-237 (2007), which is hereby incorporated by reference in its entirety), or nitrosated (Uehara, T., “Accumulation of Misfolded Protein Through Nitrosative Stress Linked to Neurodegenerative Disorders,” Antioxid. Redox. Signal 9:597-601 (2007), which is hereby incorporated by reference in its entirety) proteins, and survive amino acid limitation and (in vertebrates) process antigens (Grune et al., “Peroxynitrite Increases the Degradation of Aconitase and Other Cellular Proteins by Proteasome,” J. Biol. Chem. 273:10857-10862 (1998), which is hereby incorporated by reference in its entirety). In contrast, the functions of proteasomes in bacteria are largely unknown. Mtb is unusual among Actinomycetes in encoding only two types of chambered, ATP-dependent proteases, the proteasome and Clp (Cole et al., “Deciphering the Biology of Mycobacterium tuberculosis From the Complete Genome Sequence,” Nature 393:537-544 (1998), which is hereby incorporated by reference in its entirety). Despite a lack of information on the specific functions of the Mtb proteasome, and notwithstanding its dispensability for Mtb to survive under in vitro growth-sustaining conditions, the proteasome is indispensable for Mtb to survive in the mouse (Gandotra et al., “In vivo Gene Silencing Identifies the Mycobacterium tuberculosis Proteasome as Essential for the Bacteria to Persist in Mice,” Nat. Med. 13:1515-1520 (2007), which is hereby incorporated by reference in its entirety).

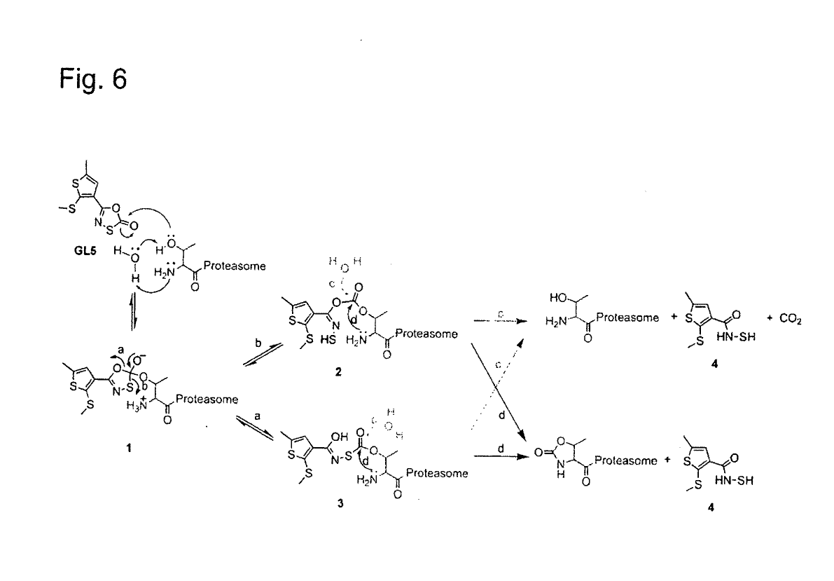

The oxathiazol-2-ones that emerged in the present invention search bear no resemblance to existing proteasome inhibitors, such as γ-lactam-β-lactones, epoxyketones, vinyl sulfones, peptidyl aldehydes, or peptidyl boronates, which all form covalent bonds with the γ-OH of the active site Thr. The LC-INAS/MS analyses of oxathiazol-2-one-treated Mtb proteasome β subunits are consistent with the mechanism of inactivation illustrated in FIG. 6. The γ-OH of the active site Thr1 attacks the carbonyl group of the oxathiazol-2-one, forming a tetrahedral intermediate 1. Intermediate 1 can undergo C—O bond cleavage (route a) or C—S bond cleavage (route b) to yield intermediate 2 or 3, respectively. The NH₂ group of the Thr1 would then attack the carbonyl group of 2 or 3 to form an oxazolidin-2-one moiety, inactivating the proteasome. In the case of the human proteasome, it was postulated that because of a different disposition of water molecules, the intermediates 2 or 3 could not undergo the nucleophilic attack by the terminal NH₂ group. Instead, they would undergo attack by the sulfhydryl or water (route c) to regenerate either the inhibitor or compound 4, with recovery of the active proteasome in both cases. Defined water molecules in the proximity of the active site Thr1 in the yeast proteasome, especially one located between the γ-OH and NH₂ of the Thr1, are involved in the hydrolysis of peptide bonds of substrates, and in the slow reactivation of eukaryotic proteasomes following inhibition by omuralide (Mc Cormack et al., “Active Site-Directed Inhibitors of Rhodococcus 20 S Proteasome. Kinetics and Mechanism,” J. Biol. Chem. 272:26103-26109 (1997); Borissenko et al., “20S Proteasome and its Inhibitors: Crystallographic Knowledge for Drug Development,” Chem. Rev. 107:687-717 (2007), which are hereby incorporated by reference in their entirety).

The unique inactivation mechanism of oxathiazol-2-ones led to an examination of selectivity of these compounds among non-proteasomal proteases, such as chymotrypsin, trypsin, cathepsin B, and matrix metalloproteinase (MMP)-2, representatives of the serine-, cysteine-, and matrix metalloproteinase classes. Most of the oxathiazol-2-ones tested were inactive or weak inhibitors of trypsin, cathepsin, B and MMP-2, with IC₅₀ values >50 μM. However, all oxathiazol-2-ones reported here except GL2 inhibited α-chymotrypsin, with IC₅₀ values ranging from 0.01-2.2 μM. GL5 reversibly inhibited α-chymotrypsin in a time-independent manner with Kᵢ 64 nM. In contrast to the proteasome's N-terminal Thr active site residue, chymotrypsin's active site Ser residue is positioned internally. Given the lack of a terminal NH₂ group, the acyl-enzyme intermediate formed by reaction of oxathiazol-2-one with the γ-OH of chymotrypsin's active site Ser can undergo either aqueous hydrolysis to reactivate the enzyme or regeneration of oxathiazol-2-one by the released —OH or —SH group attacking —CO-γO-Ser.

The differential kinetic effects of oxathiazol-2-ones on Mtb and human proteasomes and on Mtb proteasomes versus mammalian α-chymotrypsin will be advantageous in the event that oxathiazol-2 ones find eventual application in the treatment of tuberculosis. Any inhibitory activity against human proteasomes or α-chymotrypsin should be reversed following each dose, while the pathogen's proteasomes would remain inhibited. Mtb may have difficulty replacing irreversibly blocked proteasomes if bacterial protein synthesis is impaired by other chemotherapeutics or by Mtb's non-replicative state (Hu et al., “Protein Synthesis is Shutdown in Dormant Mycobacterium Tuberculosis and is Reversed by Oxygen or Heat Shock,” FEMS Microbiol. Lett. 158:139-145 (1998), which is hereby incorporated by reference in its entirety).

There is no oxathiazolone chemophore appearing in any compound that is known to inhibit a specific enzyme. Gezginci et al. found potent antimycobacterial activity in a series of pyrazines (Gezginci et al., “Antimycobacterial Activity of Substituted Isosteres of Pyridine- and Pyrazinecarboxylic Acids,” J. Med. Chem. 44:1560-1563 (2001), which is hereby incorporated by reference in its entirety), including three compounds bearing an oxathiazol-2-one moiety, but the target was not reported. The compound 5-(pyrazin-2-yl)-1,3,4-oxathiazol-2-one was synthesized, and found to inhibit the Mtb proteasome, albeit less potently than GL5.

The ability of oxathiazol-2-ones to inhibit proteasomes in intact mycobacteria and their selective toxicity for mycobacteria support the concept that the mycobacterial proteasome is both a “druggable” and an accessible target. Inhibiting macromolecular degradation rather than synthesis would be unprecedented for an anti-bacterial agent of known mechanism (Clardy et al., “Lessons From Natural Molecules,” Nature 432:829-837 (2004), which is hereby incorporated by reference in its entirety). The similarity in the concentrations of oxathiazol-2-ones required to inhibit proteasomes in mycobacteria and to kill mycobacteria is consistent with proteasome inhibition being a major mechanism for the oxathiazol-2-ones' antimycobacterial effects. However, because oxathiazol-2-ones can also inhibit α-chymotrypsin, a serine protease, they may have (an) additional target(s) in Mtb besides the proteasome. It would be a boon if, for example, the oxathiazol-2-ones inhibit not only Mtb's proteasome but also its only other chambered, ATP-dependent protease, Clp(A/X)P. Efforts are underway to diligently test this hypothesis genetically and biochemically.

Oxathiazol-2-ones reported herein are the first proteasome inhibitors found to inhibit proteasomes of Mtb by carbonlyating the γ-OH and α-NH₂ groups of the active site Thr1, thus forming a stable oxazolidin-2-one species. The much weaker and reversible inhibition of the human proteasome by the same compounds suggests that inhibitors with an oxathiazol-2-one warhead can inhibit proteasomes of different species by different modalities. The 64-fold selectivity of GL5 toward the Mtb proteasome over the human β5 proteasome subunit represents a ˜1160-fold improvement in the species selectivity ratio relative to bortezomib, a proteasome inhibitor in clinical use. Irreversible inhibition of the Mtb proteasome together with weak, reversible inhibition of human proteasomes encourages consideration of oxathiazol-2-ones for antimycobacterial drug development.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a mammalian subject for a pathogen infection, said method comprising:
administering to the subject a compound of Formula (I): 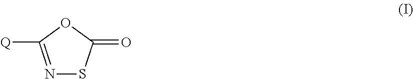
where:
Q is 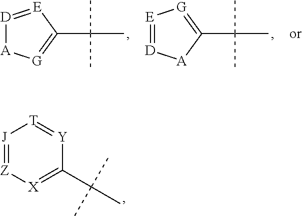
where the crossing dashed line illustrates the bond formed joining Q to the rest of the compound of Formula (I);
A is S or O;
D is N or C, wherein C is bound to R₂;
E is N or C, wherein C is bound to R₁;
G is N or C, wherein C is bound to R₃;
X is N or C, wherein C is bound to R₅;
Y is N or C, wherein C is bound to R₁;
J is N or C, wherein C is bound to R₃;
T is N or C, wherein C is bound to R₂;
Z is N or C, wherein C is bound to R₄;
wherein R₁ to R₅ are independently H, a halogen, —SR₆—NO₂, —NR₇R₈, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;
R₆ to R₁₉ are independently H, substituted or unsubstituted C₁-C₂₀ alkyl, substituted or unsubstituted C₁-C₂₀ alkenyl, substituted or unsubstituted C₁-Cₙ alkynyl, cycloalkyl, haloalkyl, heterocyclyl, or aryl,
wherein if Q is 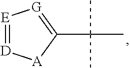
and E and D or E and G are C, R₁ and R₂, or R₁ and R₃, respectively, may form a fused substituted or unsubstituted aromatic or cyclic hydrocarbon ring and if Q is 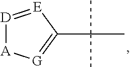
and D and E are C, R₂ and R₁ may form a fused substituted or unsubstituted aromatic ring or cyclic hydrocarbon ring, under conditions effective to treat the subject for a pathogen infection, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₈, —C(O)NHR₁₅, —C(O)NH₂, —CONR₁₆R₁₇, —CHO, —C(O)R₁₈, and —C(O)OR₁₉.

2. The method of claim 1, wherein Q is 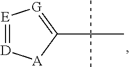
D and E are C, and R₂ and R₁ form a fused substituted or unsubstituted aromatic ring or cyclic hydrocarbon ring, whereby the compound has the following structure of Formula (II): 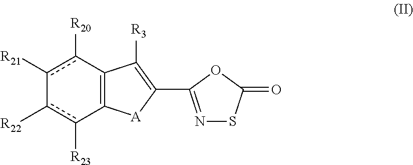
where [symbol]
is independently a single or double bond and
R₂₀ to R₂₃ are independently H, a halogen, —NO₂, —NR₇R₈, —CONR₁₆R₁₇, substituted or unsubstituted C₁-C₂₀ alkyl, substituted or unsubstituted C₁-C₂₀ alkenyl, substituted or unsubstituted C₁-C₂₀ alkynyl, cycloalkyl, heterocyclyl, or aryl.

3. The compound of claim 2, wherein the compound of Formula (II) is selected from the group consisting of:

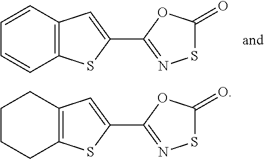

4. The method of claim 1, wherein Q is 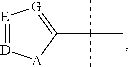
E and G are C, and R₁ and R₃ form a fused substituted or unsubstituted aromatic ring or cyclic hydrocarbon ring whereby the compound has the following structure of Formula (III):

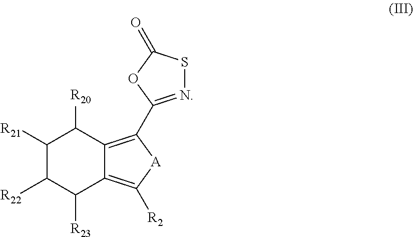

5. The method of claim 1, wherein Q is 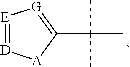
and the compound of Formula (I) has the structure of Formula (IV)

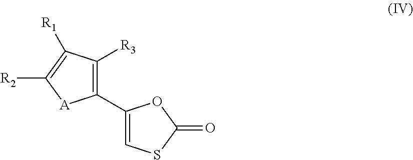

6. The compound of claim 5, wherein the compound of Formula (IV) is selected for the group consisting of

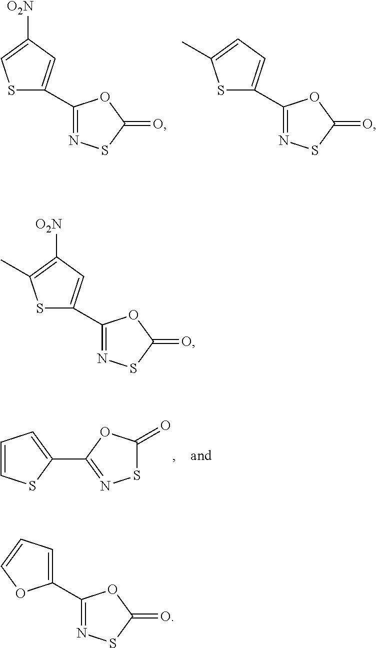

7. The method of claim 1, wherein Q is 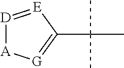
and the compound of Formula (I) has the structure of Formula (V)

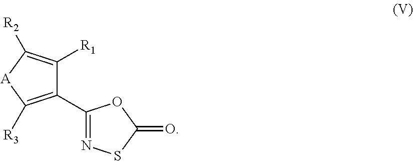

8. The compound of claim 7, wherein the compound of Formula (V) is selected from the group consisting of:

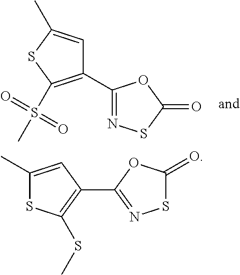

9. The method of claim 1, wherein Q is 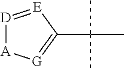
and the compound of Formula (I) is selected from the group consisting of

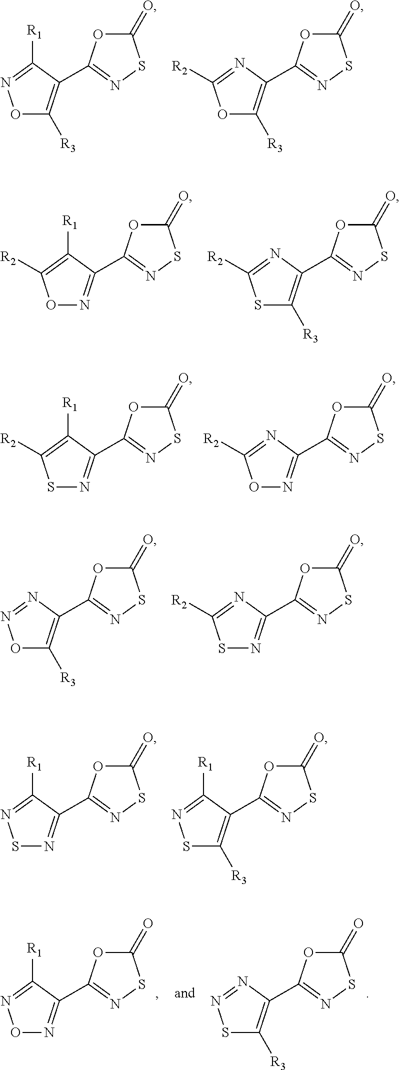

10. The method of claim 1, wherein Q is 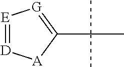
and the compound of Formula (I) is selected from the group consisting of:

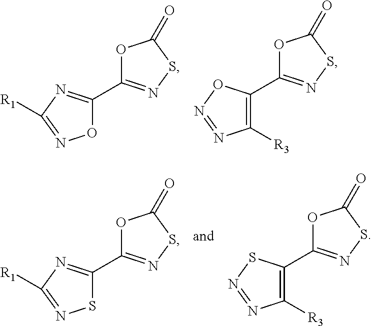

11. The method of claim 1, wherein Q is 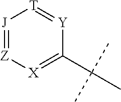
and the compound of Formula (I) has the structure of Formula (VI)

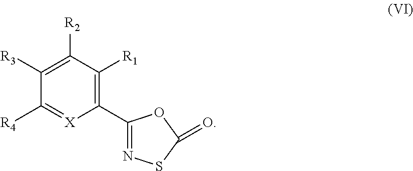

12. The compound of claim 11, wherein the compound of Formula (VI) is selected from the group consisting of 5-phenyl-1,3,4-oxathiazol-2-one, 5-(4-nitrophenyl)-1,3,4-oxathiazol-2-one, 5-(3-nitrophenyl)-1,3,4-oxathiazol-2-one, 5-(2-pyridinyl)-1,3,4-oxathiazol-2-one, 5-(3-methoxyphenyl)-1,3,4-oxathiazol-2-one, 5-(3-fluorophenyl)-1,3,4-oxathiazol-2-one, 5-(3-(trifluoromethyl)-phenyl)-1,3,4-oxathiazol-2-one, 5-(4-tolyl)-1,3,4-oxathiazol-2-one, 5-(3-tolyl)-1,3,4-oxathiazol-2-one, and 5-(3,5-dimethoxyphenyl)-1,3,4-oxathiazol-2-one.

13. The method of claim 1, wherein Q is 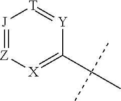
and the compound of Formula (I) is selected from the group consisting of:

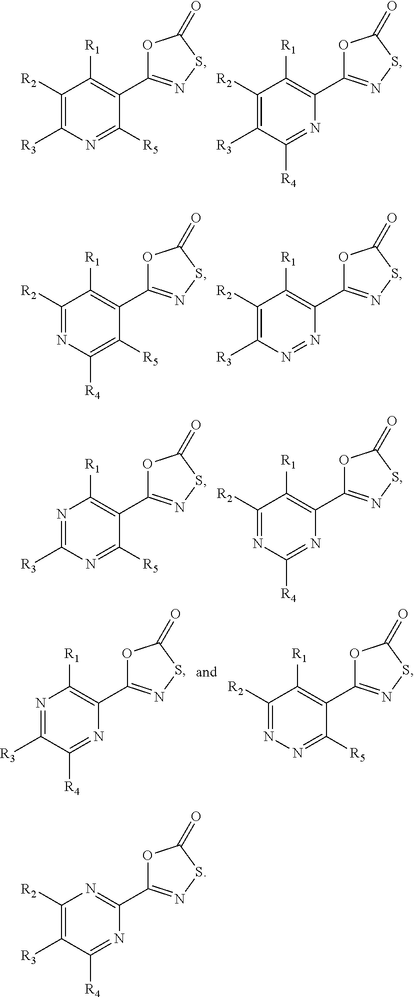

14. The method of claim 1, wherein said administering is oral, intradermal, intramuscular, intraperitoneal, intravenous, subcutaneous, or intranasal.

15. The method of claim 1, wherein the pathogen is selected from the group consisting of Mycobacterium tuberculosis, Mycobacterium leprae, and another disease-causing Mycobacterium.

16. The method of claim 1, wherein the subject is a human.

17. A method of treating a mammalian subject for cancer, said method comprising:
administering to the subject a compound of Formula (I): 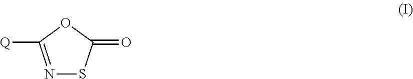
where:
Q is 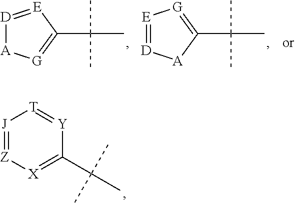
where the crossing dashed line illustrates the bond formed joining Q to the rest of the compound of Formula (I);
A is S or O;
D is N or C, wherein C is bound to R₂;
E is N or C, wherein C is bound to R₁;
G is N or C, wherein C is bound to R₃;
X is N or C, wherein C is bound to R₅;
Y is N or C, wherein C is bound to R₁;
J is N or C, wherein C is bound to R₃;
T is N or C, wherein C is bound to R₂;
Z is N or C, wherein C is bound to R₄;
wherein R₁ to R₅ are independently H, a halogen, —SR₆—NO₂, —NR₇R₈, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;
R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,
wherein if Q is 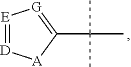
and E and D or E and G are C, R₁ and R₂, or R₁ and R₃, respectively, may form a fused substituted or unsubstituted aromatic or cyclic hydrocarbon ring and if Q is 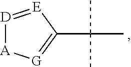
and D and E are C, R₂ and R₁ may form a fused substituted or unsubstituted aromatic ring or cyclic hydrocarbon ring, under conditions effective to treat the subject for cancer, wherein the substituted alkyl, alkenyl, alkynyl, aromatic, or cyclic hydrocarbon ring groups have substituents selected from the group consisting of hydroxyl, halogen, —CN, —NO₂, —OR₁₂, —SO₂R₉, —NH₂, —NHR₁₃, —NHSO₂R₁₄, —NR₇R₃, —C(O)NHR₁₅, —C(O)NH₂, —CONR₁₆R₁₇, —CHO, —C(O)R₁₈, and —C(O)OR₁₉.

18. The method of claim 17, wherein Q is 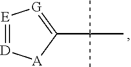
D and E are C, and R₂ and R₁ form a fused substituted or unsubstituted aromatic ring or cyclic hydrocarbon ring, whereby the compound has the following structure of Formula (II): 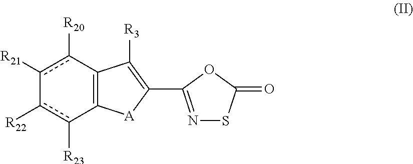
where [symbol]
is independently a single or double bond and
R₂₀ to R₂₃ are independently H, a halogen, —NO₂, —NR₇R₅, —CONR₁₆R₁₇, substituted or unsubstituted C₁-C₂₀ alkyl, substituted or unsubstituted C₁-C₂₀ alkenyl, substituted or unsubstituted C₁-C₂₀ alkynyl, cycloalkyl, heterocyclyl, or aryl.

19. The compound of claim 18, wherein the compound of Formula (II) is selected from the group consisting of:

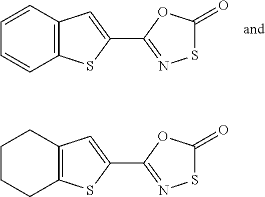

20. The method of claim 17, wherein Q is 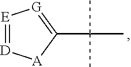
and E and G are C, R₁ and R₂ form a fused substituted or unsubstituted aromatic ring or cyclic hydrocarbon ring whereby the compound has the following structure of Formula (III):

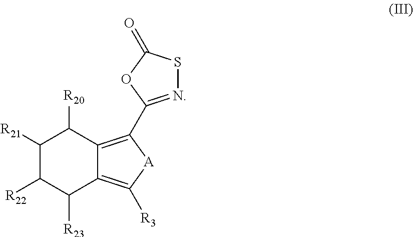

21. The method of claim 17, wherein Q is 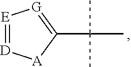
and the compound of Formula (I) has the structure of Formula (IV)

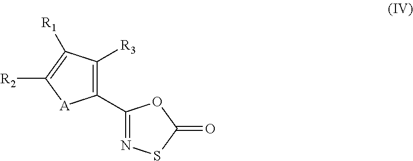

22. The compound of claim 21, wherein the compound of Formula (IV) is selected for the group consisting of:

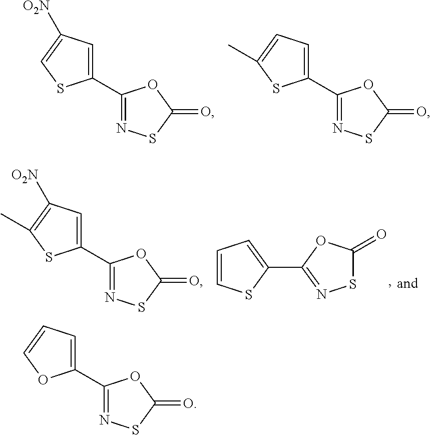

23. The method of claim 17, wherein Q is 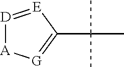
and the compound of Formula (I) has the structure of Formula (V)

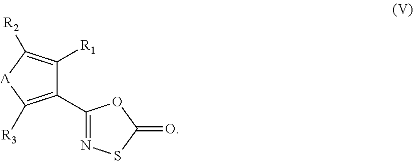

24. The compound of claim 23, wherein the compound of Formula (V) is selected from the group consisting of

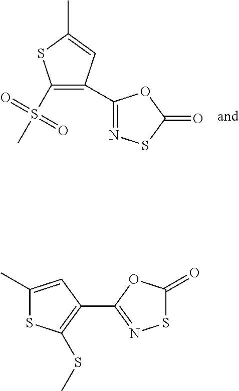

25. The method of claim 17, wherein Q is 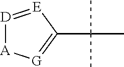
and the compound of Formula (I) is selected from the group consisting of:

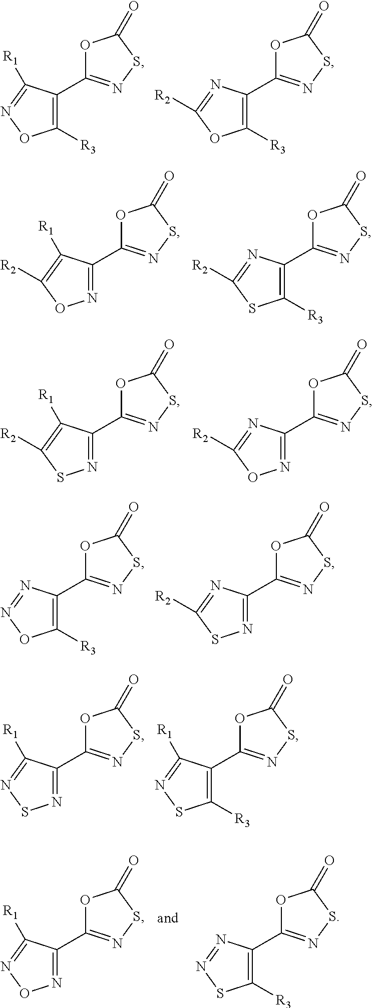

26. The method of claim 17, wherein Q is 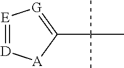
and the compound of Formula (I) is selected from the group consisting of

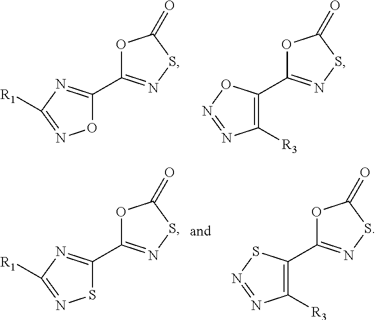

27. The method of claim 17, wherein Q is 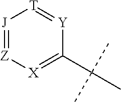
and the compound of Formula (I) has the structure of Formula (VI)

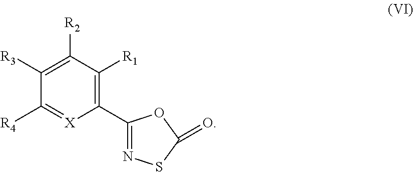

28. The compound of claim 27, wherein the compound of Formula (VI) is selected from the group consisting of 5-phenyl-1,3,4-oxathiazol-2-one, 544-nitrophenyl)-1,3,4-oxathiazol-2-one, 5-(3-nitrophenyl)-1,3,4-oxathiazol-2-one, 5-(2-pyridinyl)-1,3,4-oxathiazol-2-one, 5-(3-methoxyphenyl)-1,3,4-oxathiazol-2-one, 5-(3-fluorophenyl)-1,3,4-oxathiazol-2-one, 5-(3-(trifluoromethyl)-phenyl)-1,3,4-oxathiazol-2-one, 5-(4-tolyl)-1,3,4-oxathiazol-2-one, 5-(3-tolyl)-1,3,4-oxathiazol-2-one, and 5-(3,5-dimethoxyphenyl)-1,3,4-oxathiazol-2-one.

29. The method of claim 17, wherein Q is 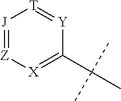
and the compound of Formula (I) is selected from the group consisting of:

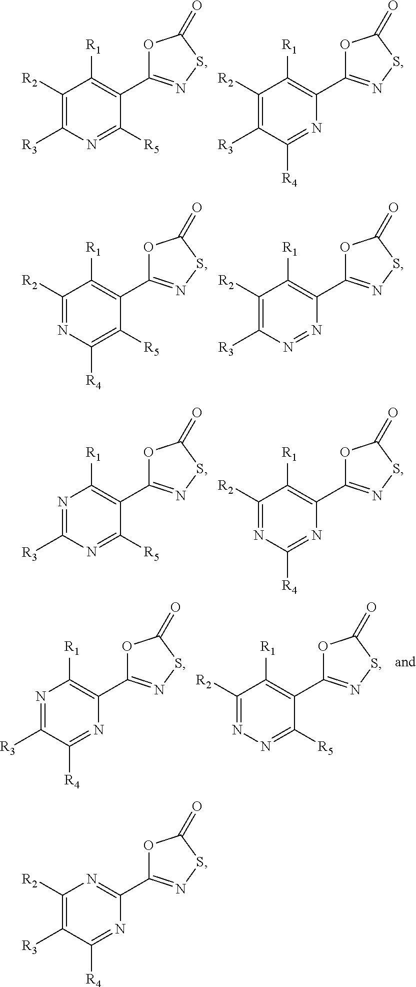

30. The method of claim 17, wherein said administering is oral, intradermal, intramuscular, intraperitoneal, intravenous, subcutaneous, or intranasal.

31. The method of claim 17, wherein the subject is a human.

32. The method of claim 17, wherein the cancer is selected from the group consisting of carcinoma, sarcoma, lymphoma, and myeloma.

33. A compound of Formula (I): 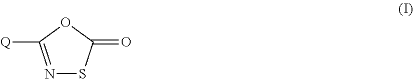
where:
Q is 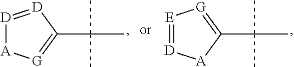
where the crossing dashed line illustrates the bond formed joining Q to the rest of the compound of Formula (I);
A is S or O;
D is N or C, wherein C is bound to R₂;
E is N or C, wherein C is bound to R₁;
G is N or C, wherein C is bound to R₃;
wherein R₁ to R₃ are independently H, a halogen, —SR₆—NO₂, —NR₇R₅, —SO₂R₉, —CONR₁₀R₁₁, —OR₁₂, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl;
R₆ to R₁₉ are independently H, substituted or unsubstituted C₁-C₂₀ alkyl, substituted or unsubstituted C₁-C₂₀ alkenyl, substituted or unsubstituted C₁-C₂₀ alkynyl, cycloalkyl, haloalkyl, heterocyclyl, or aryl,
wherein if Q is 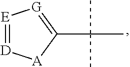
and E and D or E and G are C, R₁ and R₂, or R₁ and R₃, respectively, may form a fused substituted or unsubstituted aromatic or cyclic hydrocarbon ring and if Q is 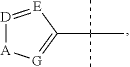
and D and E are C, R₂ and R₁ may form a fused substituted or unsubstituted aromatic or cyclic hydrocarbon ring, wherein the substituted alkyl, alkenyl, alkynyl, aromatic, or cyclic hydrocarbon ring groups have substituents selected from the group consisting of hydroxyl, halogen, —CN, —NO₂, —SR₆, —OR₁₂, —SO₂R₉, —NH₂, —NHR₁₃, —NHSO₂R₁₄, —NR₇R₈, —C(O)NHR₁₅, —C(O)NH₂, —CONR₁₆R₁₇, —CHO, —C(O)R₁₈, and —C(O)R₁₉;
with the proviso that Q is not an isoxazolyl group, and with the further proviso that at least one of D, E, or G is N if R₁ and R₂, or R₁ and R₃, do not form a fused substituted or unsubstituted aromatic hydrocarbon ring.

34. The compound claim 33, wherein Q is 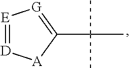
and D and E are C, R₂ and R₁ form a fused substituted or unsubstituted aromatic ring or cyclic hydrocarbon ring, whereby the compound has the following structure of Formula (II): 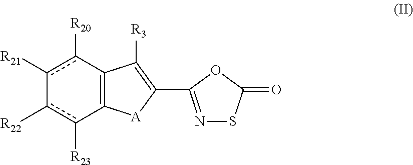
where [symbol]
is independently a single or double bond and
R₂₀ to R₂₃ are independently H, a halogen, —NO₂; —NR₇R₈, —CONR₁₆R₁₇, substituted or unsubstituted C₁-C₂₀ alkyl, substituted or unsubstituted C₁-C₂₀ alkenyl, substituted or unsubstituted alkynyl, cycloalkyl, heterocyclyl, or aryl.

35. The compound of claim 34, wherein the compound of Formula (II) is selected from the group consisting of:

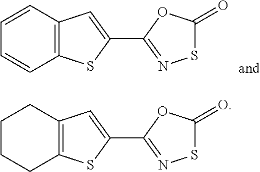

36. The compound of claim 33, wherein Q is 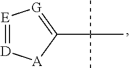
and E and G are C, R₁ and R₃ form a fused substituted or unsubstituted aromatic ring or cyclic hydrocarbon ring whereby the compound has the following structure of Formula (III):

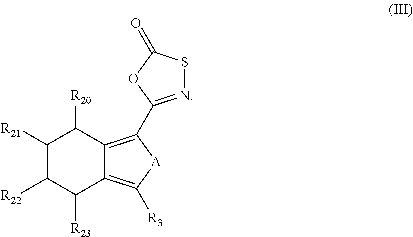

37. The compound of claim 33, wherein Q is 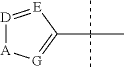
and the compound of Formula (I) is selected from the group consisting of:

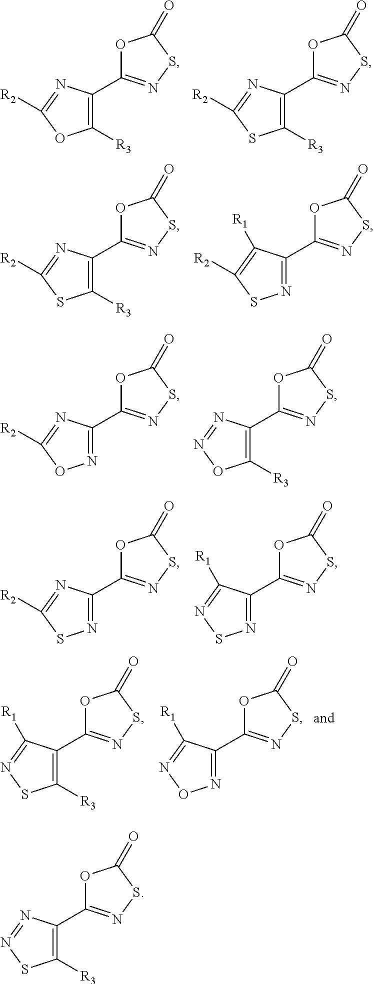

38. The compound of claim 33, wherein Q is 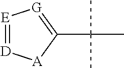
and the compound of Formula (I) is selected from the group consisting of: [chemical structure]
